SEQUENCE LISTING (13 sequences)

SEQ ID NO: 1 - protein, 607 aa, Homo sapiens
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 2 - protein, 237 aa, Artificial Sequence
  synthetic FXIS557A catalytic domain mature protein sequence
IVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPISLPSKGDRNVIYTDCWVTGWGYRKLRDKIQNTLQKAKIPLVTNEECQKRYRGHKITHKMICAGYREGGKDACKGDAGGPLSCKHNEVWHLVGITSWGEGCAQRERPGVYTNVVEYVDWILEKTQA

SEQ ID NO: 3 - DNA, 711 bp, Artificial Sequence
  Synthetic nucleotide sequence encoding FXIS557A catalytic domain mature protein sequence
atcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccataagcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagatgcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagca

SEQ ID NO: 4 - protein, 19 aa, Artificial Sequence
  Synthetic Signal peptide amino acid sequence
MGWSCIILFLVATATGVHS

SEQ ID NO: 5 - DNA, 57 bp, Artificial Sequence
  Synthetic Signal peptide nucleotide sequence
atgggctggtcctgtatcatcctctttctggtggccacagctaccggagtgcatagc

SEQ ID NO: 6 - protein, 264 aa, Artificial Sequence
  FXIS557A catalytic domain protein sequence with signal peptide and His tag
MGWSCIILFLVATATGVHSIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPISLPSKGDRNVIYTDCWVTGWGYRKLRDKIQNTLQKAKIPLVTNEECQKRYRGHKITHKMICAGYREGGKDACKGDAGGPLSCKHNEVWHLVGITSWGEGCAQRERPGVYTNVVEYVDWILEKTQAHHHHHHHH

SEQ ID NO: 7 - protein, 256 aa, Artificial Sequence
  Synthetic FXIS557A catalytic domain protein sequence with signal peptide
MGWSCIILFLVATATGVHSIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPISLPSKGDRNVIYTDCWVTGWGYRKLRDKIQNTLQKAKIPLVTNEECQKRYRGHKITHKMICAGYREGGKDACKGDAGGPLSCKHNEVWHLVGITSWGEGCAQRERPGVYTNVVEYVDWILEKTQA

SEQ ID NO: 8 - DNA, 768 bp, Artificial Sequence
  Synthetic nucleotide sequence encoding FXIS557A catalytic domain mature protein sequence and an N-terminal signal peptide of SEQ ID NO4.
atgggctggtcctgtatcatcctctttctggtggccacagctaccggagtgcatagcatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccataagcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagatgcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagca

SEQ ID NO: 9 - protein, 117 aa, Artificial Sequence
  Synthetic DEF antibody VH sequence
EVQLVQSGAEVKKPGASVKVSCKASGYTFTGYYMHWVRQAPGQGLEWMGWIDPDEGDTNYAQKFQGRVTMTRDTSISTAYMELSRLRSDDTAVYYCARLASGFRDYWGQGTLVTVSS

SEQ ID NO: 10 - protein, 108 aa, Artificial Sequence
  Synthetic DEF antibody VL sequence
DIVMTKSPSSLSASVGDRVTITCRASQGIRNDLGWYQQKPGKAPKRLIYAASSLQSGVPSRFSGSGSGTEFTLTISSLQPEDFATYYCLQHDIYASTFGPGTKVDIKR

SEQ ID NO: 11 - protein, 607 aa, Artificial Sequence
  Synthetic Full length FXIS557A protein with mutated residue at position 557
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDAGGPLSCKHNEVWHLVGITSWGEGCAQRERPGVYTNVVEYVDWILEKTQAV

SEQ ID NO: 12 - protein, 607 aa, Artificial Sequence
  Synthetic Full length FXIS557A protein sequence with mutated residue at position 482
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPISLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 13 - protein, 607 aa, Artificial Sequence
  Synthetic Full length FXIS557A protein sequence with mutated residues at positions 557 and 482
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPISLPSKGDRNVIYTDCWVTGWGYRKLRDKIQNTLQKAKIPLVTNEECQKRYRGHKITHKMICAGYREGGKDACKGDAGGPLSCKHNEVWHLVGITSWGEGCAQRERPGVYTNVVEYVDWILEKTQAV

(19) United States
(12) Patent Application Publication
(10) Pub. No.: US 2018/0216094 A1
(43) Pub. Date: Aug. 2, 2018

(54) REVERSAL AGENTS FOR FXIa INHIBITORS

(71) Applicants: THE REGENTS OF THE UNIVERSITY OF CALIFORNIA, Oakland, CA (US); PFIZER INC., New York, NY (US)

(72) Inventors: Thomas MIKITA, Sausalito, CA (US); Lauren K. ELY, Palo Alto, CA (US); Tovo DAVID, San Francisco, CA (US); Shaun R. COUGHLIN, Tiburon, CA (US)

(21) Appl. No.: 15/746,269

(22) PCT Filed: Jul. 22, 2016
(86) PCT No.: PCT/US16/43555

§ 371 Date: Jan. 19, 2018

Related U.S. Application Data
(60) Provisional application No. 62/196,085, filed on Jul. 23, 2015.

(51) Int. Cl. C12N 9/64; C07K 7/08

(52) U.S. Cl. CPC C12N 9/6443; A61K 38/00; C07K 2319/02; C07K 7/08

(57) ABSTRACT

The disclosure provides compositions and methods for counteracting the effects of direct activated Factor XI (FXIa) inhibitors in a subject by administering a variant of FXIa.

### CROSS-REFERENCE TO RELATED APPLICATIONS

This application claims priority to U.S. Provisional Application No. 62/196,085, filed Jul. 23, 2015, the entire content of which is incorporated by reference herein.

### PARTIES TO A JOINT RESEARCH AGREEMENT

The presently claimed invention was made by or on behalf of the below listed parties to a joint research agreement. The joint research agreement was in effect on or before the date the claimed invention was made and the claimed invention was made as a result of activities undertaken within the scope of the joint research agreement. The parties to the joint research agreement are PFIZER INC. and THE REGENTS OF THE UNIVERSITY OF CALIFORNIA.

### REFERENCE TO SUBMISSION OF A SEQUENCE LISTING AS A TEXT FILE

The Sequence Listing written in file SequenceListing_081906-1016798-222810PC.txt created on Jul. 21, 2016. and containing 33,601 bytes, machine format IBM-PC, MS-Windows operating system, is hereby incorporated by reference in its entirety for all purposes.

### BACKGROUND OF THE INVENTION

Factor XI is the zymogen form of the active serine protease Factor XIa (FXIa) (Ernsley, J., McEwari, P. A., and Gailani, D. Blood. 2010; 115: 2569-2577). FXIa is one of several serine proteases in the coagulation cascade, which orchestrates the clotting of blood in both normal (hemostasis) and disease (thrombotic) states. FXIa is a focus of efforts to develop novel anti-thrombotic drugs that may inhibit thrombosis with less risk of bleeding than standard agents (He, R., Chen, D., and Shilin, H. Thrombosis Research; 2012; 129, 541-550). Other serine proteases on this cascade, FXa and thrombin, are already targeted by recently approved drugs. And while these new inhibitors have many advantages over earlier vitamin K antagonists like warfarin, they still have an associated bleeding risk that limits their usage in many clinical settings (Ortel, T. L., and Arepally, G. M. Annu. Rev. Med. 2015; 66: 241-253; Flaumenhaft, R. N Engl J Med 2015. 15; 372(3):277-8). Because of this risk, efforts have more recently focused on developing reversal agents for both of these oral anticoagulants (Gostin, J., Ansell, J., Laulicht, B., Bakhru, S., and Steiner, S. PostgraciMed 2014; 126 (7): 19-24). Such reversal agents would be delivered in the case of an FXa or thrombin inhibitor-induced bleeding event.

As FXIa inhibitors come into widespread use, trauma and unscheduled surgeries in patients treated with these drugs will be inevitable. Thus, as for existing anticoagulants, a reversal agent will be needed for any future FXIa inhibitors.

Thus, there will be a need for a general reversal agent that will reverse small and large molecule FXIa inhibitors.

### BRIEF SUMMARY OF THE INVENTION

In certain aspects, the disclosure provides a modified human Factor XIa (FXIa) polypeptide comprising a catalytic domain corresponding to amino acids 370 to 606 of SEQ ID NO:1, wherein the polypeptide specifically binds a catalytic domain directed FXIa inhibitor, wherein the polypeptide contains a first mutation that reduces catalytic activity, and wherein the polypeptide exhibits reduced interactions with thrombin or a platelet receptor relative to a full-length FXIa polypeptide having the sequence of SEQ ID NO:1.

In certain aspects, the disclosure provides a modified human Factor Xla (FXIa) polypeptide comprising a catalytic domain corresponding to amino acids 370 to 606 of SEQ ID NO:1, wherein the polypeptide specifically binds a catalytic domain directed FXIa inhibitor, wherein the polypeptide contains a first mutation that reduces catalytic activity relative to a full-length FXIa polypeptide having the sequence of SEQ ID NO: 1.

In certain aspects, the disclosure provides a modified human Factor XIa (FXIa) polypeptide comprising a catalytic domain corresponding to amino acids 370 to 606 of SEQ ID NO:1, wherein the polypeptide specifically binds a catalytic domain directed FXIa inhibitor, and wherein the polypeptide contains a first mutation that reduces catalytic activity and contains a second mutation that reduces interactions with thrombin or a platelet receptor.

In certain aspects, the modified human FXIa polypeptide contains a first mutation located within the catalytic domain. In some embodiments, the first mutation inactivates the catalytic domain. In some embodiments, the first mutation is at position 557 of SEQ ID NO:1. In some embodiments, the first mutation is a serine to alanine substitution at position 557 of SEQ ID NO:1 (SEQ ID NO:11).

In certain aspects, the modified human FXIa polypeptide contains a second mutation that is located in an apple domain. In some embodiments, the modified human FXIa polypeptide comprises a plurality of apple domains and each of the apple domains comprises a mutation. In some embodiments, each of the apple domains is mutated. In some embodiments, the modified human FXIa polypeptide does not comprise apple domains.

In certain aspects, the modified human FXIa polypeptide does not interact with thrombin or platelet receptors.

In certain aspects, the modified human FXIa polypeptide does not comprise a dimerization domain. In some embodiments, the modified human FXIa polypeptide has a cysteine to serine substitution at position 482 of SEQ ID NO: 1 (SEQ ID NO:12).

In certain aspects, the disclosure provides a modified FXIa polypeptide that is substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a polypeptide having the amino acid sequence of SEQ ID NO:2. In certain aspects, the disclosure provides a modified FXIa polypeptide comprising the amino acid sequence of SEQ ID NO:2, wherein the modified FXIa polypeptide does not comprise a dimerization domain.

In certain aspects, the disclosure provides a modified FXIa polypeptide further comprising a signal peptide. In some embodiments, the signal peptide is fused to the N-terminus of the modified FXIa polypeptide. In some embodiments, the signal peptide comprises the amino acid sequence of SEQ ID NO:4.

In certain aspects, the disclosure provides a modified FXIa polypeptide that is substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a polypeptide having the amino acid sequence of SEQ ID NO:6. In certain aspects, the disclosure provides a modified FXIa polypeptide comprising the amino acid sequence of SEQ ID NO:6.

In certain aspects, the disclosure provides a modified FXIa polypeptide that is substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a polypeptide having the amino acid sequence of SEQ ID NO:7. In certain aspects, the disclosure provides a modified FXIa polypeptide comprising the amino acid sequence of SEQ ID NO:7.

In certain aspects, the disclosure provides a modified FXIa polypeptide, wherein said polypeptide has reduced procoagulant activity compared to a polypeptide comprising the amino acid sequence of SEQ ID NO:1. In some embodiments, the modified. FXIa polypeptide lacks procoagulant activity. In some embodiments, the modified FXIa polypeptide lacks anticoagulant activity.

In certain aspects, the catalytic domain directed FXIa inhibitor is a polypeptide, a peptidomimetic, an antibody, a small molecule, or a nucleic acid. In some embodiments, the catalytic domain directed FXIa inhibitor is an antibody.

In certain aspects, the catalytic domain directed FXIa inhibitor selectively binds FXIa over FXI.

In certain aspects, the disclosure provides an isolated nucleic acid molecule comprising a nucleotide sequence encoding a modified FXIa polypeptide. In some embodiments, the nucleic acid molecule is substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a polynucleotide having the sequence of SEQ ID NO:3. In some embodiments, the nucleic acid molecule comprises the nucleotide sequence of SEQ ID NO:3.

In certain aspects, the disclosure provides an isolated nucleic acid molecule comprising a nucleotide sequence encoding a modified FXIa polypeptide, wherein the nucleic acid molecule comprises the nucleotide sequence of SEQ ID NO:8. In some embodiments, the nucleic acid molecule is substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a polynucleotide having the sequence of SEQ ID NO:8.

In certain aspects, the disclosure provides a vector comprising an isolated nucleic acid molecule comprising a nucleotide sequence encoding a modified FXIa polypeptide. In some embodiments, the vector comprises the nucleotide sequence of SEQ ID NO:3 or comprises a nucleotide sequence that is substantially identical to a polynucleotide having the sequence of SEQ ID NO:3. In some embodiments, the vector comprises the nucleotide sequence of SEQ ID NO: 8 or comprises a nucleotide sequence that is substantially identical to a polynucleotide having the sequence of SEQ ID NO:8.

In certain aspects, the disclosure provides an isolated host cell comprising the vector comprising an isolated nucleic acid molecule comprising a nucleotide sequence encoding a modified FXIa polypeptide. In some embodiments, the isolated host cell comprises a vector comprising the nucleotide sequence of SEQ ID NO:3 or a nucleotide sequence that is substantially identical to a polynucleotide having the sequence of SEQ ID NO:3. In some embodiments, the isolated host cell comprises a vector comprising the nucleotide sequence of SEQ ID NO: 8 or a nucleotide sequence that is substantially identical to a polynucleotide having the sequence of SEQ ID NO:8.

In certain aspects, the disclosure provides an isolated host cell that produces a modified FXIa polypeptide.

In certain aspects, the disclosure provides a method of producing a modified FXIa polypeptide, comprising culturing an isolated host cell comprising the vector comprising an isolated nucleic acid molecule comprising a nucleotide sequence encoding a modified FXIa polypeptide under conditions that result in production of the polypeptide, and isolating the polypeptide from the host cell or culture. In some embodiments, the isolated host cell comprises a vector comprising the nucleotide sequence of SEQ ID NO:3 or a nucleotide sequence that is substantially identical to a polynucleotide having the sequence of SEQ ID NO:3. In some embodiments, the isolated host cell comprises a vector comprising the nucleotide sequence of SEQ ID NO: 8 or a nucleotide sequence that is substantially identical to a polynucleotide having the sequence of SEQ ID NO:8.

In certain aspects, the disclosure provides a method of producing a modified FXIa polypeptide, comprising culturing an isolated host cell that produces a modified FXIa polypeptide under conditions that result in production of the polypeptide, and isolating the polypeptide from the host cell or culture.

In certain aspects, the disclosure provides a method for decreasing anticoagulant activity in a subject being administered an FXIa inhibitor, comprising administering to said subject a modified FXIa polypeptide, thereby decreasing the anticoagulant activity in the subject.

In certain aspects, the disclosure provides a method for reducing clotting time in a subject being administered an FXIa inhibitor, comprising administering to said subject a modified FXIa polypeptide, thereby reducing the clotting time in the subject.

In certain aspects, the disclosure provides a modified FXIa polypeptide for use in decreasing anticoagulant activity in a subject being administered an FXIa inhibitor.

In certain aspects, the disclosure provides a modified FXIa polypeptide for use in reducing clotting time in a subject being administered an FXIa inhibitor.

In certain aspects, the disclosure provides use of a modified FXIa polypeptide in the manufacture of a medicament for decreasing anticoagulant activity in a subject being administered an FXIa inhibitor.

In certain aspects, the disclosure provides use of a modified FXIa polypeptide in the manufacture of a medicament for reducing clotting time in a subject being administered an FXIa inhibitor.

In certain aspects, the disclosure provides pharmaceutical composition comprising a modified FXIa polypeptide, and a pharmaceutically acceptable excipient.

### BRIEF DESCRIPTION OF THE DRAWINGS

FIG. 1A-B illustrate that the FXI_{S557A} catalytic domain binds with high affinity to DEF, a FXIa-blocking IgG (A), but is catalytically inactive (B).

FIG. 2 illustrates that increasing concentrations of FXI_{S557A} catalytic domain restores FXIa activity in the presence of DEF FXIa antagonist antibody.

FIG. 3 illustrates the affinity of the FXI_{S557A} catalytic domain for DEF Fab measured at 37° C. on Biacore.

FIG. 4 illustrates the reversal effects of the FXI_{S557A} catalytic domain decoy on different anti-FXIa antibodies.

FIG. 5A, FIG. 5B, and FIG. 5C illustrate that the concentrations of the FXI_{S557A} catalytic domain decoy at decoy concentrations approaching and exceeding that of the anti-FXIa antibody DEF reverse the effects of DEF in human plasma in an FXIIa-triggered thrombin generation assay.

FIG. 6A-6C show the results of APTT and PT assays in which the FXI_{S557A} catalytic domain decoy molecule is administered. FIG. 6A and FIG. 6B illustrate that the FXI_{S557A} catalytic domain decoy molecule reverses effects of anti-FXIa antibody DEF in human plasma in an in vivo rabbit dosing experiment. FIG. 6C illustrates that control rabbits dosed with decoy molecule but not DEF do not exhibit changes in coagulation.

### DETAILED DESCRIPTION OF THE INVENTION

### I. INTRODUCTION

Factor XI (“FXI”) is the zymogen of a serine protease, Factor XIa (“FXIa”), that is activated during coagulation as part of the coagulation cascade. FXI is a homodimer in which each subunit contains four apple domains (A1-A4) and a catalytic domain (CD). In the FXI homodimer, the site of contact between the FXI subunits is within the A4 domain, and the catalytic domain rests atop the apple domains. The apple domains contain binding sites for a number of ligands, including the FXIa substrate FIX. FXI subunits are activated by cleavage of a bond between A4 and the catalytic domain. See, Gailani et al., J. Thromb Haemost., 2009, 7 (Suppl 1):75-78, incorporated by reference herein.

The development of large macromolecule inhibitors of FXIa activity, such as the novel high affinity, high potency, high selectivity, and fast acting IgG inhibitors of the coagulation cascade serine protease FXIa as described in U.S. Provisional Patent Application “Antibodies to Coagulation Factor XIa and Uses Thereof,” No. 62/196,037, incorporated by reference in its entirety herein, exacerbates the need for reversal reagents, as these anticoagulants will have a much longer half-life compared to their small molecule counterparts. Disclosed herein are FXIa decoy molecules that act as general reversal agents to reverse small and large molecule FXIa inhibitors. The disclosed FXIa decoy molecules may decrease anticoagulant activity in a subject being administered an FXIa inhibitor.

The disclosed FXIa decoy molecules can be used in combination with an FXIa inhibitor in the prevention, treatment, and/or amelioration of diseases, disorders or conditions caused by and/or associated with FXIa activity. Such diseases, disorders or conditions include, but are not limited to, acute major bleeding caused by trauma; acute major bleeding during surgery or other type of interventional procedure; other acute bleeding while on an FXIa inhibitor; thrombotic or thromboembolic diseases; atrial fibrillation (AF) or thromboembolism related to atrial fibrillation (Afib); venous thromboembolism (VTE); VTE in the medically ill; Afib in the renal disease population and/or patients identified as being at high risk for bleeding; acute coronary syndromes; use of extracorporeal circulations and devices in which blood contacts artificial surfaces; myocardial infarction; congestive heart failure; acute myocardial infarction; pulmonary embolism; thrombosis; deep vein thrombosis; renal vein thrombosis; transient ischemic attack; thrombotic stroke; thromboembolic stroke; cardiogenic thromboembolism; atherosclerosis; inflammatory diseases; pulmonary hypertension; pulmonary and/or hepatic fibrosis; and sepsis; among others, as would be appreciated by one skilled in the art provided with the teachings disclosed herein. Additional uses include situations in which blood touches artificial surfaces, including mechanical heart valves, extracorporeal circulations, left ventricular assist devices, and catheters, wires, and other devices introduced in to the heart and blood vessels. Examples of diseases and disorders are provided in WO 2013/167669, incorporated herein by reference.

In one aspect, the novel FXIa decoy molecules disclosed herein comprise a modified monomer of the catalytic domain of FXIa. In some embodiments, these decoy molecules contain a first mutation that reduces catalytic activity, a deletion that reduces interactions with other coagulation factors, and a third mutation that prevents dimerization. In another aspect, methods of making FXIa decoy molecules, compositions comprising these molecules, and methods of using these molecules are provided.

### II. DEFINITIONS

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology. immunology, microbiology, genetics and protein and nucleic acid chemistry and hybridization described herein are those well-known and commonly used in the art.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, NY (2003); Short Protocols in Molecular Biology (Wiley and Sons, 1999); immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the terms “FXIa decoy molecule” and “FXIa variant” refer to a modified polypeptide that presents a Factor Xia (FXIa)-like surface for binding FXIa-specific inhibitors and that has reduced catalytic activity and reduced ability to create dysfunction in the normal coagulation process by interaction with other coagulation factors (e.g., thrombin or a platelet receptor) via abrogation of native FXI/FXIa functions. As used herein, an “FXIa-like surface” refers to a surface that mimics FXIa for binding antibodies or other FXIa inhibitors that bind to the FXIa catalytic domain (e.g., that bind to the active site or that bind to the catalytic domain outside of the active site). In some embodiments, the FXIa decoy molecule or FXIa variant presents a surface that mimics FXIa (e.g., mimics the tertiary structure of FXIa) for binding antibodies or other FXIa inhibitors that bind to the active site and that has additional FXIa surface features outside the active site. In some embodiments, the FXIa decoy molecule or FXIa variant presents a surface for binding antibodies or other inhibitors that act by binding to the FXIa catalytic domain outside of the active site. In some embodiments, the FXIa decoy molecule or FXIa variant comprises a substitution at a residue in the catalytic domain (e.g., in the active site in the catalytic domain or in a residue outside of the active site in the catalytic domain) that reduces or eliminates the catalytic activity of the FXIa decoy molecule or FXIa variant, but that otherwise mimics the surface of FXIa for binding antibodies or other FXIa inhibitors at the FXIa catalytic domain. In some embodiments, the FXIa decoy molecule or FXIa variant is a modified human FXIa polypeptide and has reduced catalytic activity and reduced ability to create dysfunction in the normal coagulation process relative to a human FXIa polypeptide having the sequence of SEQ ID NO:1. An FXIa decoy molecule or FM variant of the disclosure may be produced by any technique for expressing a protein.

The term “reduced catalytic activity,” as used with respect to an FXIa decoy molecule or FXIa variant, means that the FXIa decoy molecule or FXIa variant has decreased protease activity as compared to a wild-type (naturally-occurring) FXIa protein, e.g., a protein of SEQ ID NO:1 that has been converted from the zymogen to the active form. In some embodiments, an FXIa decoy molecule or FXIa variant has reduced catalytic activity if the protease activity of the FXIa decoy molecule or FXIa variant is decreased by at least about 10%, 20%, 30%, 40%, 50%, 60%, 70%, 80%, 90% or more relative to a wild-type FXIa protein. In some embodiments, an FXIa decoy molecule or FXIa variant has reduced catalytic activity if the protease activity of the FXIa decoy molecule or FXIa variant is below detectable levels. Methods of measuring the catalytic activity of FXIa are known in the art and include, but are not limited to, a fluorogenic peptide substrate cleavage assay or a thrombin generation assay. In some embodiments, catalytic activity is measured by a fluorogenic peptide substrate cleavage assay as described in the Examples section below. In some embodiments, catalytic activity is measured by measuring thrombin generation in human plasma.

The term “reduced interactions with thrombin or a platelet receptor,” as used with respect to an FXIa decoy molecule or FXIa variant, means that the FXIa decoy molecule or FXIa variant has a decreased binding affinity for thrombin or for a platelet receptor as compared to a wild-type (naturally-occurring) FXIa protein, e.g., an FXIa protein having the sequence of SEQ ID NO:1. In some embodiments, an FXIa decoy molecule or FXIa variant exhibits reduced interactions with thrombin or a platelet receptor if the binding affinity of the FXIa decoy molecule or FXIa variant for thrombin or a platelet receptor is decreased by at least about 10%, 20%, 30%, 40%, 50%, 60%, 70%, 80%, 90% or more relative to a wild-type FXIa protein, e.g., an FXIa protein having the sequence of SEQ ID NO: 1. In some embodiments, an FXIa decoy molecule or FXIa variant does not detectably bind to or interact with thrombin or a platelet receptor. Methods of measuring specific binding and binding affinity are known in the art and include, but are not limited to, solid-phase binding assays, immunoprecipitation, surface plasmon resonance (e.g., Biacore™ (GE Healthcare, Piscataway, N.J.)), kinetic exclusion assay, fluorescence-activated cell sorting (FACS), Octet™ (ForteBio, Inc., Menlo Park, Calif.), examining substrate competition for thrombin, examining binding of radiolabelled decoy to platelet membranes, and examining the effect of the decoy on coagulation under conditions in which FXI activation by thrombin may contribute to activation of the cascade.

As used herein, the term “FXI_{S557}” refers to an FXIa decoy molecule or FXIa variant comprising an alanine substitution at a position corresponding to position 557 of SEQ ID NO:1 (SEQ ID NO:11). The term “FXI_{S557A}” is not limited by the protein sequence set forth in SEQ ID NO:2. Rather this term additionally includes the variety of isoforms and homologous proteins described herein with the specified substitution mutations corresponding to position 557 of the primary sequence that behave as catalytically dead FXIa decoys to reverse the effects of an anti-FXIa antibody but do not interfere with the coagulation process.

As used herein, the term “catalytic domain directed FXIa inhibitor” refers to an inhibitor of activated FXI (FXIa) that specifically binds a portion of the catalytic domain of FXIa (e.g., a portion of amino acids 370-606 of SEQ ID NO:1) or specifically binds an epitope within the catalytic domain of FXIa. In some embodiments, the inhibitor binds at or near the FXIa active site within the catalytic domain. The structure of the FM active site is described in the art, e.g., in Jin et al., Journal of Biological Chemistry, 2005, 280:4704-4712, and includes a cavity containing a catalytic triad that is composed of the residues His314, Asp462, and Ser557 (as numbered with reference to SEQ ID NO: 1, which represents the protein sequence of the mature FXI zymogen lacking an N-terminal signal peptide). In some embodiments, the inhibitor binds at the cavity containing the catalytic triad. In some embodiments, the catalytic domain directed FXIa inhibitor preferentially binds FXIa over FXI. In some embodiments, a catalytic domain directed FXIa inhibitor is a polypeptide, a peptidomimetic, a protein, an antibody, an antigen-binding fragment, a small molecule, or a nucleic acid. In some embodiments, the catalytic domain directed FXIa inhibitor is an antibody.

As used herein, an “isolated protein,” “isolated polypeptide,” or “isolated variant” is a protein, polypeptide or variant that by virtue of its origin or source of derivation (1) is not associated with naturally associated components that accompany it in its native state, (2) is free or substantially free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein may also be rendered substantially free of naturally-associated components by isolation, using protein purification techniques well known in the art.

A protein or polypeptide is “substantially pure,” “substantially homogeneous,” or “substantially purified” when at least about 60%, e.g., at least about 60 to 75% of a sample exhibits a single species of polypeptide. The polypeptide or protein may be monomeric or multimeric. A substantially pure polypeptide or protein will typically comprise about 50%, 60%, 70%, 80% or 90% (w/w) of a protein sample, more usually about 95%, and may be over 99% pure. Protein purity or homogeneity may be indicated by a number of means known in the art, such as polyacrylarnide gel electrophoresis of a protein sample, followed by visualizing a single polypeptide band upon staining the gel with a stain well known in the art. For certain purposes, higher resolution may be provided by using HPLC or other means known in the art for purification.

The term “residue,” as used herein, refers to a position in a protein or polypeptide and its associated amino acid identity. For example, Asparagine 297 (also referred to as Asn297, also referred to as N297) is a residue in the human antibody IgG1.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

As known in the art, “polynucleotide,” or “nucleic acid,” as used interchangeably herein, refers to a chain of nucleotides of any length, and includes DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR⁻, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, a molecule “preferentially binds” or “specifically binds” (used interchangeably herein) to a cell or to a substance (e.g., a protein, polypeptide, or antibody, e.g., a protein, polypeptide, or antibody comprising an epitope) if the molecule reacts or associates more frequently, more rapidly, with greater duration, and/or with greater affinity with a particular cell or substance than it does with alternative cells or substances. It will be understood by a person of ordinary skill in the art reading this definition, for example, that a molecule (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specific binding” or “preferential binding,” as used herein, also refers to a compound or substance, e.g., a protein, a nucleic acid, an antibody, and the like, that recognizes and binds to a specific molecule, but does not substantially recognize or bind other molecules in a sample. For instance, a protein or a peptide receptor that recognizes and hinds to a cognate ligand or binding partner in a sample, but that does not substantially recognize or bind other molecules in the sample, specifically binds to that cognate ligand or binding partner. Thus, under designated assay conditions, the specified binding moiety binds preferentially to a particular target molecule and does not bind in a significant amount to other components present in a test sample.

A variety of assay formats may be used to select a protein that specifically binds a molecule of interest. For example, solid-phase ELISA immunoassay, immunoprecipitation, Biacore™ (GE Healthcare, Piscataway, N.J.), kinetic exclusion assay (KinExA®, Sapidyne Instruments, Inc., Boise, Id.)), fluorescence-activated cell sorting (FACS), Octet™ (ForteBio, Inc., Menlo Park, Calif.) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background.

The term “binding affinity” is herein used as a measure of the strength of a non-covalent interaction between two molecules. The term “binding affinity” is used to describe monovalent interactions (intrinsic activity).

Binding affinity between two molecules through a monovalent interaction may he quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D}) is related to kₐ and k_{d} through the equation K_{D}=k_{d}/kₐ. The value of the dissociation constant can be determined directly by well-known methods, and can be computed even for complex mixtures by methods such as those, for example, set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of ligands are known in the art, including for example, ELISAs, Western blots, RIAs, and flow cytometry analysis, and other assays exemplified elsewhere herein. The binding kinetics and binding affinity of the protein also can be assessed by standard assays known in the art, such as Surface Plasmon Resonance (SPR), e.g. by using a Biacore™ system, or KinExA.

A competitive binding assay can be conducted in which the binding of the protein to the target is compared to the binding of the target by another ligand of that target, such as another protein or a soluble receptor that otherwise binds the target. The concentration at which 50% inhibition occurs is known as the Kᵢ. Under ideal conditions, the Kᵢ is equivalent to K_{D}. The Kᵢ value will never be less than the K_{D}, so measurement of Kᵢ can conveniently he substituted to provide an upper limit for K_{D}.

Following the above definition, binding affinities associated with different molecular interactions, e.g., comparison of the binding affinity of different proteins for a given ligand, may be compared by comparison of the K_{D} values for the individual protein/ligand complexes. K_{D} values for proteins or other binding partners can be determined using methods well established in the art. One method for determining the K_{D} is by using surface plasmon resonance, typically using a biosensor system such as a Biacore® system.

Similarly, the specificity of an interaction tray be assessed by determination and comparison of the K_{D} value for the interaction of interest, e.g., a specific interaction between a protein and a ligand, with the K_{D} value of an interaction not of interest, e.g., a control protein known not to bind anti-FXIa antibodies.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected and/or transformed in vivo with a polynucleotide of this disclosure.

As used herein, “treatment” is an approach for obtaining beneficial or desired clinical results. For purposes of this disclosure, beneficial or desired clinical results include, but are not limited to, one or more of the following: improved survival rate (reduced mortality), reduction in thrombosis or thromboembolism (antibody), reduction in bleeding (decoy), decreased extent of damage from the disease, decreased duration of the disease, and/or reduction in the number, extent, or duration of symptoms related to the disease. The term includes the administration of the compounds or agents of the present disclosure to prevent or delay the onset of the symptoms, complications, or biochemical indicia of a disease, alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

“Ameliorating” means a lessening or improvement of one or more symptoms as compared to not administering an FXIa decoy molecule. “Ameliorating” also includes shortening or reduction in duration of a symptom.

As used herein, an “effective dosage” or “effective amount” of drug, compound, or pharmaceutical composition is an amount sufficient to affect any one or more beneficial or desired results. In more specific aspects, an effective amount prevents, alleviates or ameliorates symptoms of disease or infection, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more symptoms of a factor FXIa-mediated disease, disorder or condition, decreasing the dose of other medications required to treat the disease, enhancing the effect of another medication, and/or delaying the progression of the disease of patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, an “individual” or a “subject” is a mammal, in some embodiments, a human. Mammals also include, but are not limited to, farm animals (e.g., cows, pigs, horses, chickens, etc.), sport animals, pets, primates, horses, dogs, cats, mice and rats. In some embodiments, the FXIa decoy molecules described herein decrease anticoagulant activity in a subject being administered an FXIa inhibitor. In some embodiments, the individual is considered to be at risk for a disease, disorder or condition mediated by or associated with Factor XIa. In certain embodiments, the subject has a thrombotic disease, disorder or condition, such as deep vein thrombosis.

As used herein, “vector” means a construct, which is capable of delivering, and, in some embodiments, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. In some embodiments, diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10.

Unless otherwise defined, all technical and scientific terms used herein have the same meaning as commonly understood by one of ordinary skill in the art to which this disclosure belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Exemplary methods and materials are described herein, although methods and materials similar or equivalent to those described herein can also be used in the practice or testing of the present invention. The materials, methods, and examples are illustrative only and not intended to be limiting.

### III. FXIa DECOY MOLECULES

In one aspect, compositions and methods for counteracting the anticoagulant effect of a direct FXIa inhibitor in a subject in need thereof are provided. As described herein, Applicants have discovered that certain FXIa variants rapidly and completely counteract the effect of a direct FXIa inhibitor in a dose dependent manner. Applicants have discovered that such an FXIa variant, or FKla decoy molecule, was able to bind anti-FXIa antibodies that inhibit FXIa activity (for example, anti-FXIa antibodies such as those described in U.S. Provisional Patent Application “Antibodies to Coagulation Factor XIa and Uses Thereof,” No. 62/196,037). Applicants have further demonstrated that the binding activity exhibited by the FXIa decoy molecule towards anti-FXia antibodies allowed the decoy molecule to function as a reversal agent reversing the effects of the anti-FXIa antibodies. Applicants' data disclosed herein suggest that the decoy molecules of the disclosure are likely to be effective against any modality, antibody, peptide, or small molecule inhibitor that acts by binding at or near the FXIa active site or, more broadly, to the FXIa protease domain.

In some embodiments, an FXIa decoy molecule or FXIa variant binds to an anti-FXIa antibody described in U.S. Provisional Patent Application No. 62/196,037 (“Antibodies to Coagulation Factor XIa and Uses Thereof”). In some embodiments, the anti-FXIa antibody (described in U.S. Provisional Patent Application “Antibodies to Coagulation Factor XIa and Uses Thereof,” No. 62/196,037) has the VH sequence of the antibody DEF as shown below:

(SEQ ID NO: 9)
EVQLVQSGAEVKKPGASVKVSCKASGYTFTGYYMHWVRQAPGQGLEWMGW
IDPDEGDTNYAQKFQGRVTMTRDTSISTAYMELSRLRSDDTAVYYCARLA
SGFRDYWGQGTLVTVSS.

In some embodiments, the anti-FXIa antibody (described in U.S. Provisional Patent Application “Antibodies to Coagulation Factor XIa and Uses Thereof,” No. 62/196,037) has the VL sequence of the antibody DEF as shown below:

(SEQ ID NO: 10)
DIVMTKSPSSLSASVGDRVTITCRASQGIRNDLGWYQQKPGKAPKRLIYA
ASSLQSGVPSRFSGSGSGTEFTLTISSLQPEDFATYYCLQHDIYASTFGP
GTKVDIKR.

In some embodiments, the FXIa decoy molecules of the present disclosure present an FXIa-like surface for binding FXIa-specific inhibitors (e.g., catalytic domain directed. FXIa inhibitors). In some embodiments, the FXIa decoy molecule is a modified full-length Factor XIa (FXIa) polypeptide having at least one mutation (e.g., substitution or deletion) relative to a wild-type (naturally occurring) FXIa polypeptide. In some embodiments, the FXIa decoy molecules of the disclosure are modified FXIa polypeptides comprising a catalytic domain corresponding to amino acids 370 to 606 of SEQ ID NO:1, which specifically bind a catalytic domain directed FXIa inhibitor, and which contain at least one mutation (e.g., substitution or deletion) that reduces catalytic activity. In some embodiments, the FXIa decoy molecules of the disclosure are modified FXIa polypeptides comprising a catalytic domain corresponding to amino acids 370 to 606 of SEQ ID NO:1, which specifically bind a catalytic domain directed FXIa inhibitor, and which contain a first mutation that reduces catalytic activity and a second mutation that reduces interactions with thrombin or a platelet receptor.

In some embodiments, the first mutation in the FXIa decoy molecule is located within the catalytic domain. In some embodiments, an FXIa decoy molecule is an FXIa protein comprising an amino acid substitution that makes the decoy catalytically inactive compared to a wild-type FXIa protein in VIVO or in vitro. In some embodiments, the first mutation in the FXIa decoy molecule is at the amino acid corresponding to amino acid 557 of SEQ ID NO:1 (SEQ ID NO:11). Examples of FXIa decoy molecules that are useful in methods of the disclosure are variants comprising a modification where the serine (Ser) residue at position 557 of SEQ ID NO:1 is substituted with alanine (Ala). Such decoy molecules lack catalytic activity. In some embodiments, loss of catalytic activity prevents procoagulant effects or unexpected activities.

In some embodiments, the second mutation of the FXIa decoy molecule is located in an apple domain corresponding to one of the four 50 kDa apple domains of the polypeptide of SEQ ID NO:1. In some embodiments, the FXIa decoy molecule comprises at least one mutation in each of one or more (e.g., two or more) apple domains. In some embodiments, each of the apple domains of the FXIa decoy molecule is mutated.

In some embodiments, the FXIa decoy molecule lacks cross-factor interacting apple domains. Thus, in some embodiments, the FXIa decoy molecule is a modified truncated Factor XIa. (FXIa) polypeptide, relative to a wild-type (naturally occurring) FXIa polypeptide, wherein the FXIa decoy molecule comprises a mutation that reduces catalytic activity and wherein the FXIa decoy molecule exhibits reduced or decreased interactions with thrombin or a platelet receptor relative to a wild-type FXIa polypeptide, for example by virtue of a truncation or deletion of the one or more apple domains.

In certain such embodiments, the FXIa decoy molecule lacks the ability to create dysfunction in the normal coagulation process by interaction with other coagulation factors via abrogation of native FM/FXIa functions (e.g., interactions with high molecular weight kininogen, Factor IX, thrombin and platelet receptors; Emsley J, McEwan P A, Gailani D. Blood, 2010; 115(13):2569-77). In some embodiments, it is desirable that the decoy not interfere with the function of endogenous FXI/FXIa in the normal coagulation process, such as via the presence of functional apple domains. In some embodiments, the FXIa decoy molecule does not interact with thrombin or platelet receptors.

In some embodiments, the FXIa decoy molecule lacking apple domains lacks the ability to dimerize. In some embodiments, the FXIa decoy molecule does not comprise a dimerization domain. Without wishing to be bound by theory, reducing dimerization capability or mutating the apple domains could impact proper formation/conformation of the catalytic subunit. In some embodiments, the FXIa decoy molecule comprises a modification at a residue corresponding to position 482 of SEQ ID NO:1. In some embodiments, the FXIa decoy molecule comprises a modification where the Cys at position 482 is Ser. In such embodiments, the FXIa decoy lacks the ability to form a disulfide bond at position 482. In some embodiments, the FXIa decoy molecule comprises the amino acid sequence of SEQ ID NO:2, wherein the FXIa decoy molecule does not comprise a dimerization domain.

The FXla decoy molecules can be variants of any mammalian FXIa. In some embodiments, the FXIa decoy molecule is a variants of human FXIa (e.g., a human FXIa polypeptide having the sequence of SEQ ID NO:1). In some embodiments, the decoy FXIa molecule comprises the amino acid sequence identified in SEQ ID NO:2. In some embodiments, the decoy FXIa molecule comprises the amino acid sequence identified in SEQ ID NO:6. In some embodiments, the decoy FXIa molecule comprises the amino acid sequence identified in SEQ ID NO:7. In some embodiments, the FXIa decoy molecule is substantially identical (e.g., at least 70%, at least 75%, at least 80%, at least 85%, at least 90%, at least 91%, at least 92%, at least 93%, at least 94%, at least 9.5%, at least 96%, at least 97%, at least 98%, or at least 99% identical) to the sequence of any of SEQ ID NO:2, SEQ ID NO:6, or SEQ ID NO:7.

In some embodiments, a FXIa decoy molecule that is substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to the sequence of any of SEQ ID NO:2, SEQ ID NO:6, or SEQ ID NO:7 has reduced catalytic activity as compared to the FXIa polypeptide of SEQ ID NO:1. In some embodiments, the FXIa decoy molecule lacks detectable catalytic activity. In some embodiments, the catalytic activity or lack of detectable catalytic activity of the FXIa decoy is determined via a fluorogenic peptide substrate cleavage assay.

In certain embodiments, the FXIa decoy molecules of the disclosure may be further modified by incorporating a signal peptide. The teen “signal peptide” or “signal peptide sequence” is defined herein as a peptide sequence usually present at the N-terminal end of newly synthesized secretory or membrane polypeptides which directs the polypeptide across or into a cell membrane of the cell (the plasma membrane in prokaryotes and the endoplasmic reticulum membrane in eukaryotes). It is usually subsequently removed. In particular, the signal peptide may be capable of directing the polypeptide into a cell's secretory pathway. Signal peptides are well understood by those of skill in the art and may include any known signal peptide that is compatible with the cell that will express the FXIa decoy molecule. See, U.S. Pat. No. 8,859,254, incorporated herein by reference in its entirety. In some embodiments, the signal peptide is incorporated at the N-terminus of the protein. Processing of the FXIa decoy molecule by signal peptidase produces the decoy as the mature protease domain. In some embodiments, the signal peptide is directly fused to the N-terminus of SEQ ID NO:2. In some embodiments, the signal peptide comprises the amino acid sequence identified in SEQ ID NO:4. In some embodiments, the FXIa decoy molecule modified by the signal peptide at the N-terminus of the protein comprises the amino acid sequence identified in SEQ ID NO:6. In some embodiments, the FXIa decoy molecule modified by the signal peptide at the N-terminus of the protein comprises the amino acid sequence identified in SEQ ID NO:7.

In some embodiments, the present disclosure relates to a novel decoy molecule that comprises a monomer of the catalytic domain of FXIa, and this molecule comprises (1) an amino acid substitution that changes the catalytic serine residue of the active site to an alanine, rendering FXIa catalytically dead, (2) an amino acid substitution that replaces a cysteine with a serine so as to prevent unwanted disulfide formation, and (3) a molecule that connects a signal peptide directly to the mature N-terminus of the FXIa catalytic (protease) domain, thereby removing the FXI apple domains and unwanted interactions with other proteins and allowing processing by a signal peptidase to produce the protein as the mature protease domain.

In some embodiments, the decoy FXIa molecule is modified by a His tag at the carboxy terminus. In some embodiments, the His tag facilitates purification or detection of the protein. In some embodiments, the His tag labeled-decoy FXIa, molecule comprises the amino acid sequence identified in SEQ ID NO:6.

In certain embodiments, FXIa variants of the disclosure are derived from FXI variant preproteins comprising native wild-type human signal sequence and/or propeptide sequence. In other embodiments, FXI signal sequences and/or propeptide from non-human species can be used in place of the corresponding native amino acid sequences. And in yet other embodiments, signal sequence and/or propeptide sequence from other human or non-human secreted proteins can be used in place of the corresponding native amino acid sequences.

According to yet other embodiments, isoforms of FXIa variant proteins, including those specific variants described in the preceding paragraphs, can include isoforms in which a variable number of amino acids (for example, 1, 2, 3, 4, 5, 6, or more amino acids) are either missing from or added to the carboxy or N-termini of the of the protein. In certain embodiments, the FXIa variant protein comprises amino acids 370 to 605, 370 to 604, 370 to 603, 370 to 602, 370 to 601, 370 to 600, or 370 to 599 of SEQ ID NO:1.

According to certain embodiments, FXIa variants of the disclosure include proteins with a certain minimal degree of homology or sequence identity compared to the amino acid sequence of wild-type FXIa in SEQ ID NO:1. Thus, for example, FXIa, variants include proteins that are at least 60%, 70%, 80%, 85%, 90%, 95%, 98%, or 99% homologous or identical in sequence with the wild-type FXIa in SEQ ID NO:1, wherein such FXIa, variants also include a substitution at the amino acid position corresponding to position 482 of SEQ ID NO:1 with Ser, and/or a substitution at the amino acid position corresponding to position 557 of SEQ ID NO:1 with Ala. In certain embodiments, an FXIa variant includes up to 10, 9, 8, 7, 6, 5, 4, 3, 2, or 1 conservative amino acid substitution in the catalytic domain. In certain embodiments, an FXIa variant has less than 5, 4, 3, or 2 conservative amino acid substitutions in the catalytic domain. Percentage amino acid sequence homology or identity can readily be determined using software such as Protein BLAST available at the website of the National Center for Biotechnology Information (blast.ncbi.nlm.nih.gov/Blast.cgi).

According to other non-limiting embodiments, FXIa decoy molecules of the disclosure can also include FXIa molecules containing one or more post-translational modifications including, without limitation, one or more O-linked or N-linked carbohydrate groups or a variable number of gamma-carboxyglutamic acid (Gla) residues. FXIa decoy molecules of the disclosure can further include chemically modified FXIa decoy molecule proteins. Other FXIa decoy molecules useful in the methods of the disclosure are also possible.

According to other non-limiting embodiments, FXIa decoy molecules of the disclosure can also include amino- and/or carboxyl-terminal fusions ranging in length from one residue to polypeptides containing a hundred or more residues, as well as intrasequence insertions of single or multiple amino acid residues. Examples of terminal insertions include an N-terminal methionyl residue or the FXIa decoy molecule fused to an epitope tag. Other insertional variants of the FXIa decoy molecule include the fusion to the N- or C-terminus of the FXIa decoy molecule of an enzyme or a polypeptide which increases the half-life of the FXIa decoy molecule in the blood circulation.

If desired, the polynucleotide sequence FXIa decoy molecules of interest may be cloned into a vector for expression or propagation. The sequence encoding the FXIa decoy molecules of interest may be maintained in vector in a host cell and the host cell can then be expanded and frozen for future use. Production of recombinant FXIa decoy molecules in cell culture can be carried out through means known in the art.

### IV. POLYNUCLEOTIDES, VECTORS, AND HOST CELLS

In another aspect, polynucleotides encoding any of the FXIa decoy molecules described herein and methods of making any of the polynucleotides described herein are provided. Polynucleotides can be made and expressed by procedures known in the art. In some embodiments, the disclosure provides polynucleotides or compositions, including pharmaceutical compositions, comprising polynucleatides, encoding any of the foIlowing FXIa decoy molecules: SEQ ID NO:2, SEQ ID NO:6, and SEQ ID NO:7. In some embodiments, these polynucleotides comprise the following sequences: SEQ ID NO:3 and/or SEQ ID NO:8.

In another aspect, the disclosure provides polynucleotides and variants thereof encoding an FXIa decoy molecules, wherein such variant polynucleotides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to any of the specific nucleic acids disclosed herein (e.g., at least 70% to SEQ ID NO:3 or SEQ ID NO:8). These amounts are not meant to be limiting, and increments between the recited percentages are specifically envisioned as part of the disclosure.

Polyrnucleotides complementary to any such sequences are also encompassed by the present disclosure. Thus, in some embodiments, a polynucleotide is complementary to a polynucleotide encoding an FXIa decoy molecule of SEQ ID NO:2, SEQ ID NO:6, or SEQ ID NO:7 or is complementary to a polynucleotide variant having at least 70% (e.g., at least 75%, at least 80%, at least 85%, at least 87%, at least 89%, at least 90%, at least 91%, at least 92%, at least 93%, at least 94%, at least 95%, at least 96%, at least 97%, at least 98%, or at least 99%) sequence identity to a polynucleotide encoding an FXIa decoy molecule of SEQ ID NO:2, SEQ ID NO:6, or SEQ ID NO:7. In some embodiments, a polynucleotide is complementary to a polynucleotide having the sequence of SEQ ID NO:3 or SEQ ID NO:8 or is complementary to a polynucleotide variant having at least 70% (e.g., at least 75%, at least 80%, at least 85%, at least 87%, at least 89%, at least 90%, at least 91%, at least 92%, at least 93%, at least 94%, at least 95%, at least 96%, at least 97%, at least 98%, or at least 99%) sequence identity to a polynucleotide having the sequence of SEQ ID NO:3 or SEQ ID NO:8. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules and/or support materials.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be onducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol, Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence)

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC 19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest and/or the polynucleotides themselves, can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The disclosure also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). In some embodiments, the host cells express the cDNAs at a level of about 5 fold higher, in some embodiments. 10 fold higher, and in some embodiments, 20 fold higher than that of the corresponding endogenous antibody or protein of interest, if present, in the host cells. Screening the host cells for a specific binding to FXIa is effected by an immunoassay or FACS. A cell overexpressing the antibody or protein of interest can be identified.

An expression vector can be used to direct expression of an FXIa decoy molecule. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to the sympathetic trunk or ganglion, or into a coronary artery, atrium, ventrical, or pericardium.

Targeted delivery of therapeutic compositions containing an expression vector, or subgenomic polynucleotides can also be used. Receptor-mediated DNA delivery techniques are described in, for example, Findeis et al., Trends Biotechnol., 1993, 11:202; Chiou et al., Gene Therapeutics: Methods And Applications Of Direct Gene Transfer, J. A. Wolff, ed., 1994; Wu et al., J. Biol. Chem., 1988, 263:621; Wu et al., J. Biol. Chem., 1994, 269:542; Zenke et al., Proc. Natl. Acad. Sci. USA, 1990, 87:3655; Wu et al., J. Biol. Chem., 1991, 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy, 1994, 1:51; Kimura Human Gene Therapy, 1994, 5:845; Connelly, Human Gene Therapy, 1995, 1:185; and Kaplitt Nature Genetics, 1994, 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; NATO 94/03622; WO 93/25698; WO 93/25234; WO 93/11230; WO 93/10218; WO 91/02805; U.S. Pat. Nos. 5, 219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250, ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther., 1992, 3:147); ligand-linked DNA (see, e.g., Wu, J. Biol. Chem., 1989, 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 1994, 14:2411, and in Woffendin, Proc. Natl. Acad. Sci., 1994, 91:1581.

### V. METHODS OF USE

In one aspect, the methods of the disclosure are useful to counteract a direct FXIa inhibitor. As used herein, a “direct FXIa inhibitor” is an inhibitor that binds directly to FXIa and selectively binds FXIa over other proteases.

According to the disclosure, an FXIa variant can be used to counteract a direct FXIa inhibitor that binds FXIa. The activity of an FXIa decoy molecule can be confirmed by bioassays known to test the targeted biological activities. Some of the methods for characterizing FXIa decoy molecules are described in detail in the Examples. Non-limiting exemplary tests include a fluorogenic peptide substrate assay and a thrombin generation assay. Other tests are also possible within the knowledge of those of ordinary skill in the art.

The disclosure encompasses the use of an FXIa variant to counteract direct FXIa inhibitors, including but not limited to protein inhibitors, antibody inhibitors, synthetic inhibitors, small molecule inhibitors, orally available inhibitors, or reversible inhibitors. The FXIa inhibitor may be any combination of these features, such as an orally available, synthetic, reversible, small molecule inhibitor. In certain embodiments, the direct FXIa inhibitor binds to the catalytic domain of FXIa. In certain embodiments, the direct FXIa inhibitor binds to the active site of the catalytic domain. In certain embodiments, the direct FXIa inhibitors may be selected from IgG inhibitors of the coagulation cascade serine protease FXIa as described in U.S. Provisional Patent Application “Antibodies to Coagulation Factor XIa and Uses Thereof,” No. 62/196,037, being filed concurrently herewith and incorporated by reference in its entirety herein.

According to some embodiments, reversing the effects of a direct FXIa inhibitor in a sample by administering an FXIa decoy molecule reduces the activity of a direct FXIa inhibitor in the sample. In some embodiments, treatment with an FXIa decoy molecule reduces activity of the direct FXIa inhibitor in a sample at least 10%, 20%, 30%, 40%, 50%, 60%, 70%, 80%, 90%. 95%, or 99% in the presence of a direct FXIa inhibitor compared to absence of treatment with a FXIa decoy molecule. In other embodiments, treatment with an FXIa decoy molecule reduces the activity of a direct FXIa inhibitor in a sample about 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, or 95%-100%. These amounts are not meant to be limiting, and increments between the recited amounts are specifically envisioned as part of the disclosure.

According to some embodiments, reversing the effects of a direct FXIa inhibitor in a sample by administering an FXIa decoy molecule increases the amount of thrombin produced in the sample. In some embodiments, treatment with an FXIa decoy molecule increases thrombin production in a subject at least 10%, 20%, 30%, 40%, 50%, 60%, 70%, 80%, 90%, 95%, 100%, 1.5 fold, 2-fold, 3-fold, 4-fold, 5-fold, 6-fold, 7-fold, 10-fold, 15-fold, 20-fold, 25-fold, 30-fold, at least 50-fold, or more in the presence of a direct FXIa inhibitor compared to the absence of an FXIa decoy molecule. Thrombin production in a sample can be determined using the thrombin generation assay (TGA) or other technique familiar to those of ordinary skill in the art. These amounts are not meant to be limiting, and increments between the recited amounts are specifically envisioned as part of the disclosure.

According to some embodiments, reversing the effects of a direct FXIa inhibitor in a sample by administering an FXIa decoy molecule increases the amount of FXIa enzymatic activity observed in a fluorogenic substrate assay in the sample. In some embodiments, treatment with an FXIa decoy molecule increases enzymatic cleavage of a fluorogenic substrate in a sample at least 10%, 20%, 30%, 40%, 50%, 60%, 70%, 80%, 90%, 95%, 100%, 1.5 fold, 2-fold, 3-fold, 4-fold, 5-fold, 6-fold, 7-fold, 10-fold, 15-fold, 20-fold, 25-fold, 30-fold, at least 50-fold, or more in the presence of a direct FXIa inhibitor compared to the absence of an FXIa decoy molecule. These amounts are not meant to be limiting, and increments between the recited amounts are specifically envisioned as part of the disclosure.

According to the methods of the disclosure, an FXIa decoy molecule, such as FXI_{S557A}, is administered to a subject whose blood contains a direct FXIa inhibitor. In some embodiments, an FXIa decoy molecule of the disclosure can be administered to a subject to reverse the effects of a direct FXIa inhibitor where such inhibitor occurs at therapeutic concentrations. In other embodiments, an FXIa decoy molecule of the disclosure can be administered to a subject to reverse the effects of a direct FXIa inhibitor where such inhibitor occurs at supratherapeutic concentrations. A supratherapeutic concentration is one that is higher than that ordinarily considered required to safely achieve anti-coagulation in a particular subject or class of subjects. Supratherapeutic concentrations of a direct FXIa inhibitor can result from accidental or intentional overdose. Supratherapeutic concentrations of a direct FXIa inhibitor can also result from unexpected effects in particular subjects, such as an unexpectedly high sensitivity to these drugs, or unexpectedly slow rate of clearance, due for example to drug interactions or other factors. Determination of what would be a therapeutic concentration or supratherapeutic concentration of direct FXIa inhibitor in a particular subject or class of subjects is within the knowledge of those ordinarily skilled in the art.

According to the disclosure, an FXIa decoy molecule is used to counteract a direct FXIa inhibitor or inhibitors that selectively bind FXIa over other trypsin-like proteases by at least 5-fold, at least 6-fold, at least 7-fold, at least 10-fold, at least 15-fold, at least 20-fold, at least 25-fold, at least 30-fold, at least 50-fold, at least 100-fold, at least, 500-fold, at least 1,000-fold, at least 5,000-fbld or at least 10,000-fold. These amounts are not meant to be limiting, and increments between the recited amounts are specifically envisioned as part of the disclosure.

The direct FXIa inhibitor may bind an FXIa, decoy molecule with a Kᵢ of about 2×10⁻⁷ M or less. “Kᵢ” refers to the inhibitor constant of a particular inhibitor-target interaction, which is the concentration required to produce half maximum inhibition. One can determine the Kᵢ by using methods known in the art. The disclosure contemplates, thus, counteracting a direct FXIa inhibitor that binds an FXIa decoy molecule with a Kᵢ of about 2×10⁻⁸ M or less, about 1×10⁻⁸ M or less, about 9×10⁻⁹ M or less, about 8×10⁻⁹ M or less, about 7×10⁻⁹ M or less, about 6×10⁻⁹ M or less, about 5×10⁻⁹ M or less, about 4×10⁻⁹ M or less, about 3×10⁻⁹ M or less, about 2×10⁻⁹ M or less, about 1×10⁻⁹ M or less, about 9×10⁻¹⁰ M or less, about 8×10⁻¹⁰ M or less, about 7×10⁻¹⁰ M or less, about 6×10⁻¹⁰ M or less, about 5×10⁻¹⁰ M or less, about 4×10⁻¹⁰ M or less, about 3×10⁻¹⁰ M or less, about 2×10⁻¹⁰ M or less, about 1 ×10⁻¹⁰ M or less, about 9×10⁻¹¹ M or less, about 8×10⁻¹¹ M or less, about 7×10⁻¹¹ M or less, about 6×10¹¹ M or less, about 5×10⁻¹¹ M or less, about 4×10⁻¹¹ M or less, about 3×10⁻¹¹ M or less, about 2×10⁻¹¹ M or less, about 1×10⁻¹¹ M or less, about 9×10⁻¹² M or less, about 8×10¹² M or less, about 7×10⁻¹² M or less, about 6×10⁻¹² M or less, about 5×10⁻¹² M or less, about 4×10⁻¹² M or less, about 3×10⁻¹² M or less, about 2×10⁻¹² M or less, or about 1×10⁻¹² M or less, or less. The direct FXIa inhibitor to be counteracted by an FXIa decoy molecule according to the methods of the disclosure may bind a wild-type FXIa with a Kᵢ at least 1.5 fold, at least 2-fold, at least 3-fold, at least 4-fold, at least 5-fold, at least 6-fold, at least 7-fold, at least 10-fold, at least 15-fold, at least 20-fold, at least 25-fold, at least 30-fold, or at least 50-fold less than it binds the FXIa decoy molecule. The direct FXIa inhibitor may bind an FXIa dimer complex comprising a wild-type FXIa with about the same Kᵢ. These amounts are not meant to be limiting, and increments between the recited amounts are specifically envisioned as part of the disclosure.

In one aspect, the disclosure provides methods for counteracting the effects of a direct FXIa inhibitor in a subject who is bleeding (internally or externally) or is at risk of bleeding (e.g., in the course of a planned surgery) by administering an FXIa decoy molecule. In some embodiments, the direct FXIa inhibitor may be present in the subject at a therapeutic concentration or a higher concentration (i.e., a supratherapeutic concentration). In some embodiments, the therapeutic concentration may be an overdose in sensitive individuals. The methods of the disclosure, thus, are useful for providing an antidote to an overdose of a direct FXIa inhibitor. In various embodiments, the subject of treatment may be a human or a veterinary subject.

Direct inhibitor overdose can be detected based on existence of symptoms or signs of excessively reduced clotting ability. Non-limiting examples include evidence of gastrointestinal bleeding, including dark tarry stools, bloody stools, and vomiting of blood. Other examples include nosebleeds, and increased tendency to, or severity of, bruising or bleeding from minor cuts and scrapes.

In a clinical setting, direct inhibitor overdose can be detected directly or by measuring the ability of subject blood to clot and detecting deviations from the expected degree of anti-coagulation. Blood clotting potential can be measured in ways familiar to those ordinarily skilled in the art. For example, overdose may be suspected when a subject's activated partial thromboplastin time (aPTT) is prolonged, suggesting the presence of a factor Xla inhibitor. aPTT prolongation over control values may be 1.5, 2.0, 2.5 or greater.

The FXIa decoy molecule may be administered whenever it is desired to counteract the effects of the direct FXIa inhibitor, including but not limited to before a planned surgery, after an injury resulting in external or internal bleeding, or after a direct FXIa inhibitor overdose. According to the disclosure, the FXIa decoy molecule may be administered at least about 12 hours, at least about 6 hours, at least about 3 hours, at least about 2 hours, at least about 1 hour, at least about 30 minutes, at least about 10 minutes, or at least about 5 minutes of when the desired counteracting effect is needed, such as before a planned surgery, after an injury resulting in external or internal bleeding or after a direct FXIa inhibitor overdose.

According to another embodiment, the disclosure provides a method of administering an FXIa decoy molecule to effect the urgent reversal of acquired coagulopathy due to FXIa inhibition therapy in a subject with acute major bleeding. In some embodiments, subjects are adult human patients. In other embodiments, subjects are pediatric human patients.

In some embodiments, acute major bleeding is caused by trauma. In other embodiments, acute major bleeding occurs during surgery or other type of interventional procedure. Exemplary non-limiting interventional procedures include dental extractions, incisions, drainage, vascular surgery, appendectomy, herniotomy or hernioplasty, abdominal surgery, cholecystectomy, trephination (burr hole), lumbar puncture, cardiac pacemaker insertion, hip fracture surgery, uterine, prostate and bladder surgery, and others. In yet other embodiments, acute major bleeding can be spontaneous bleeding with no apparent cause.

Without limitation, sites of acute major bleeding include gastrointestinal bleeding, subcutaneous or intramuscular bleeding, bladder bleeding, hemarthrosis, subdural hematoina, nasal bleeding, peritoneal bleeding, uterine bleeding, and other sites of bleeding.

Effective treatment with FXIa decoy molecules of the disclosure can reverse the effects of a direct FXIa inhibitor. Successful reversal of such effects by an FXIa decoy molecule can be determined in a variety of ways and be measured or monitored using different assays, methods, or endpoints.

In some embodiments, treatment with an FXIa decoy molecule to reverse the effects of a direct FXIa inhibitor is monitored using tests or assays performed on blood or plasma from a subject treated with an FXia decoy molecule. A blood sample can be taken from a subject at a predetermined time after treatment with an FXIa decoy molecule. The blood, or plasma prepared from it, is then subjected to one or more tests to determine if certain hemostatic pharmacodynamic parameters have been normalized despite the presence of direct FXIa inhibitor. If normalization is found then the subject need not be further treated with an FXIa decoy molecule. If normalization is not found, however, then further treatment with an FXIa decoy molecule in accordance with the methods of the disclosure may be required to reverse the effect of a direct FXIa inhibitor. Tests for monitoring the effectiveness of treatment with an FXIa decoy molecule include tests that directly or indirectly measure the ability to clot or that measure the activity of a direct FXIa inhibitor. Non-limiting exemplary tests include activated partial thromboplastin time, partial thromboplastin time, fluorogenic peptide substrate assay, thromboelastometry, thromboelastography, thrombin generation assay, level of prothrombin fragment 1+2, and level of thrombin-antithrombin III complex. Other tests are also possible within the knowledge of those of ordinary skill in the art.

According to some embodiments, reversing the effects of a direct FXIa inhibitor in a subject by administering an FXIa decoy molecule reduces bleeding in the subject. In some embodiments, treatment with an FXIa decoy molecule reduces bleeding in a subject at least 10%, 20%, 30%, 40%, 50%, 60%, 70%, 80%, 90%, 95%, or 99% in the presence of a direct FXIa inhibitor compared to absence of treatment with an FXIa decoy molecule. In other embodiments, treatment with an FXIa decoy molecule reduces bleeding in a subject about 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, or 95%100%. These amounts are not meant to be limiting, and increments between the recited amounts are specifically envisioned as part of the disclosure.

According to some embodiments, reversing the effects of a direct FXIa inhibitor in a subject by administering an FXIa decoy molecule reduces the activity of a direct FXIa inhibitor in the subject. In some embodiments, treatment with an FXIa decoy molecule reduces activity of the direct FXIa inhibitor in a subject at least 10%, 20%, 30%, 40%, 50%, 60% 70%, 80%, 90%, 95%, or 99% in the presence of a direct FXIa inhibitor compared to absence of treatment with an FXIa decoy molecule. In other embodiments, treatment with an FXIa decoy molecule reduces the activity of a direct FXIa inhibitor in a subject about 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, or 95%-100%. These amounts are not meant to be limiting, and increments between the recited amounts are specifically envisioned as part of the disclosure.

According to some embodiments, reversing the effects of a direct FXIa inhibitor in a subject by administering an FXIa decoy molecule increases the amount of thrombin produced in the blood or plasma of the subject. In some embodiments, treatment with an FXIa decoy molecule increases thrombin production in a subject at least 10%, 20%, 30%, 40%, 50%, 60%, 70%, 80%, 90%, 95%, 100%, 1.5 fold, 2-fold, 3-fold, 4-fold, 5-fold, 6-fold, 7-fold, 10-fold, 15-fold, 20-fold, 25-fold, 30-fold, at least 50-fold, or more in the presence of a direct FXIa inhibitor compared to the absence of an FXIa decoy molecule. Thrombin production in the blood or plasma of a subject can be determined using the thrombin generation assay (TGA) or other technique familiar to those of ordinary skill in the art. These amounts are not meant to be limiting, and increments between the recited amounts are specifically envisioned as part of the disclosure.

According to some embodiments, reversing the effects of a direct FXIa inhibitor in a subject by administering an FXIa decoy molecule increases the amount of FXIa enzymatic activity observed in a fluorogenic substrate assay in a sample of blood or plasma of a subject. In some embodiments, treatment with an FXIa decoy molecule increases enzymatic cleavage of a fluorogenic substrate in a sample at least 10%, 20%, 30%, 40%, 50%, 60%, 70%, 80%, 90%, 95%, 100%, 1.5 fold, 2-fold, 3-fold, 4-fold, 5-fold, 6-fold, 7-fold, 10-fold, 15-fold, 20-fold, 25-fold, 30-fold, at least 50-fold, or more in the presence of a direct FXIa inhibitor compared to the absence of an FXIa decoy molecule. These amounts are not meant to be limiting, and increments between the recited amounts are specifically envisioned as part of the disclosure.

According to some embodiments, reversing the effects of a direct FXIa inhibitor in a subject by administering an FXIa decoy molecule increases clotting in the subject. In some embodiments, treatment with an FXIa decoy molecule increases clotting in a subject at least 10%, 20%, 30%, 40%, 50%, 60%, 70%, 80%, 90%, 95%, 100%, 1.5 fold, 2-fold, 3-fold, 4-fold, 5-fold, 6-fold, 7-fold, 10-fold, 15-fold, 20-fold, 25-fold, 30-fold, at least 50-fold, or more in the presence of a direct FXIa inhibitor compared to the absence of an FXIa decoy molecule. These amounts are not meant to be limiting, and increments between the recited amounts are specifically envisioned as part of the disclosure.

According to some embodiments, reversing the effects of a direct FXIa inhibitor in a subject by administering an FXIa decoy molecule reduces clotting time in the subject. In some embodiments, treatment with an FXIa decoy molecule reduces clotting time in a subject at least 10%, 20%, 30%, 40%, 50%, 60%, 70%, 80%, 90%, 95%, or 99% in the presence of a direct FXIa inhibitor compared to absence of treatment with an FXIa decoy molecule. In other embodiments, treatment with an FXIa decoy molecule reduces clotting time in a subject about 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, or 95%-100%. These amounts are not meant to be limiting, and increments between the recited amounts are specifically envisioned as part of the disclosure.

According to some embodiments, clotting time is determined by measuring the subject's activated partial thromboplastin time (aPTT), which decreases as hemostasis is restored. aPTT is the amount of time it takes for plasma to clot after addition of an intrinsic pathway activator such as ellagic acid or kaolin. Patients treated with a factor XIa inhibitor will typically have an APTT more than 1.5-fold longer than control and will often have an aPTT more than 2.0-fold longer than control. Treatment with a decoy will reverse this prolongation to less than 1.5-fold control and more typically to less than 1.2 fold control.

aPTT can be measured at a predetermined time after administration of an FXIa decoy molecule. Thus, in some non-limiting embodiments, aPTT is measured 15 mins, 20 mins, 30 mins, 40 mins, 45 mins, 50 mins, 60 mins or more after administration of an FXIa decoy molecule. Other times are also possible according to the knowledge of those of ordinary skill in the aft.

In yet other embodiments, the methods of thromboelastometry or thromboelastography may be used to analyze clot formation or clotting time.

According to some embodiments, reversing the effects of a direct FXIa inhibitor in a subject by administering an FXIa decoy molecule increases the level of prothrombin fragment 1+2 (PFI+2) in the blood or plasma of the subject. In some embodiments, treatment with FXIa decoy molecule increases PFT+2 in a subject at least 10%, 20%, 30%, 40%, 50%, 60%, 70%, 80%, 90%, 95%, 100%, 1.5-fold, 2-fold, 3-fold, 4-fold, 5-fold, 6-fold, 7-fold, 10-fold, 15-fold, 20-fold, 25-fold, 30-fold, at least 50-fold, or more in the presence of a direct FXIa inhibitor compared to the absence of an FXla decoy molecule. These amounts are not meant to be limiting, and increments between the recited amounts are specifically envisioned as part of the disclosure.

According to some embodiments, reversing the effects of a direct FXIa inhibitor in a subject by administering an FXIa decoy molecule increases the level of thrombin-antithrombin III complex (TAT) in the blood or plasma of the subject. In some embodiments, treatment with an FXIa decoy molecule increases TAT in a subject at least 10%, 20%, 30%, 40%, 50%, 60%, 70%, 80%, 90%, 95%, 100%, 1.5-fold, 2-fold, 3-fold, 4-thld, 5-fold, 6-fold, 7-fold, 10-fold, 15-fold, 20-fold, 25-fold, 30-fold, at least 50-fold, or more in the presence of a direct FXIa inhibitor compared to the absence of an FXIa decoy molecule. These amounts are not meant to be limiting, and increments between the recited amounts are specifically envisioned as part of the disclosure.

According to some embodiments, reversing the effects of a direct FXIa inhibitor in a subject by administering an FXIa decoy molecule reduces activated partial thromboplastin time (aPTT) in the subject. In some embodiments, treatment with an FXIa decoy molecule reduces activated partial thromboplastin time (aPTT) in a subject at least 10%, 20%, 30%, 40%, 50%, 60%, 70%, 80%, 90%, 95%, or 99% in the presence of a direct FXIa inhibitor compared to absence of treatment with an FXIa decoy molecule. In other embodiments, treatment with an FXIa decoy molecule reduces aPTT in a subject about 5%-10%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, or 95%-100%. These amounts are not meant to be limiting, and increments between the recited amounts are specifically envisioned as part of the disclosure.

In other embodiments, clinical endpoints can be relied upon to determine if hemostasis has been adequately restored in a subject treated with an FXIa decoy molecule to reverse the effects of a direct FXIa inhibitor. For example, where a subject presents with acute bleeding, clinical hemostatic efficacy can be scored “very good” where prompt cessation of existing bleeding occurs after treatment with an FXIa decoy molecule; “satisfactory” where there is a 1-2 hr delay in bleeding cessation; “questionable” where there is a >2 hr delay in bleeding cessation; and “none” where an effect on bleeding is absent. Where treatment with an FXIa decoy molecule is determined to be less than satisfactory, then an additional dose of FXIa decoy molecule can be administered to effect adequate hemostasis. In a further example, where a subject is undergoing an interventional procedure, clinical hemostatic efficacy can be scored “very good” where nonnal hemostasis is attained during the procedure, “satisfactory” where intraprocedural hemostasis is mildly abnormal as judged by quantity or quality of blood loss (e.g., slight oozing); “questionable” where intraprocedural hemostasis is moderately abnormal as judged by quantity or quality of blood loss (e.g., controllable bleeding); and “none” where intraprocedural hemostasis is severely abnormal as judged by quantity or quality of blood loss (e.g., severe refractory hemorrhage).

In some embodiments, the FXIa decoy molecules disclosed herein act as general reversal agents to reverse small and large molecule FXIa inhibitors. The disclosed FXIa decoy molecules decrease anticoagulant activity in a subject being administered an FXIa, inhibitor. The disclosed FXIa decoy molecules can be used in combination with an FXIa inhibitor in the prevention, treatment, and/or amelioration of diseases, disorders or conditions caused by and/or associated with FXIa activity. Such diseases, disorders or conditions include, but are not limited to, acute major bleeding caused by trauma; acute major bleeding during surgery or other type of interventional procedure; thrombotic or thromboembolic diseases; atrial fibrillation (AF) or thromboembolism related to atrial fibrillation (Afib); venous thromboembolism (VTE); VTE in the medically ill; Afib in the renal disease population and/or patients previously identified as bleeders; acute coronary syndromes; use of extracorporeal circulations and devices in which blood contacts artificial surfaces; myocardial infarction; congestive heart failure; acute myocardial infarction; pulmonary embolism; thrombosis; deep vein thrombosis; renal vein thrombosis; transient ischemic attack; thrombotic stroke; thromboembolic stroke; cardiogenic thromboembolism; atherosclerosis; inflammatory diseases; pulmonary hypertension; pulmonary and/or hepatic fibrosis; and sepsis; among others, as would be appreciated by one skilled in the art provided with the teachings disclosed herein. Additional uses include situations in which blood touches artificial surfaces, including mechanical heart valves, extracorporeal circulations, left ventricular assist devices, and catheters, wires, and other devices introduced in to the heart and blood vessels. Examples of diseases and disorders are provided in WO 2013/167669, incorporated herein by reference.

In certain aspects, the disclosure provides for a method for decreasing anticoagulant activity in a subject being administered an FXIa inhibitor, comprising administering to said subject an FXIa decoy molecule as described herein, wherein the anticoagulant activity is reduced compared with the anticoagulant activity in the subject prior to administration of the polypeptide. In certain aspects, the disclosure provides for a method for reducing clotting time in a subject being administered an FXIa inhibitor, comprising administering to said subject an FXIa decoy molecule as described herein, wherein the clotting time is reduced compared with the clotting time in the subject prior to administration of the polypeptide.

In some embodiments, the disclosure provides an FXIa decoy molecule as described herein for use in decreasing anticoamilant activity in a subject being administered an FXIa inhibitor. In some embodiments, the disclosure provides an FXIa decoy molecule as described herein for use in reducing clotting time in a subject being administered an FXIa inhibitor.

In some embodiments, the disclosure provides use of an FXIa decoy molecule as described herein in the manufacture of a medicament for decreasing anticoagulant activity in a subject being administered an FXIa inhibitor. In some embodiments, the disclosure provides use of an FXIa decoy molecule as described herein in the manufacture of a medicament for reducing clotting time in a subject being administered an FXIa inhibitor.

### VI. DOSING AND ADMINISTRATION

A therapeutically effective dose of a direct FXIa inhibitor depends upon numerous factors that are well known to a medical practitioner of skill in the art. In some embodiments, a typical therapeutic dose of the high affinity, high potency, high selectivity, and fast acting IgG inhibitors of the coagulation cascade serine protease FXIa as described in U.S. Provisional Patent Application No. 62/196,037 (“Antibodies to Coagulation Factor XIa and Uses Thereon”), incorporated by reference in its entirety herein, is about 100 nM. However, according to the disclosure, an FXIa decoy molecule can be administered to counteract lower or higher concentrations of inhibitor. The dose of the high affinity, high potency, high selectivity, and fast acting IgG inhibitors of the coagulation cascade serine protease FXIa as described in U.S. Provisional Patent Application “Antibodies to Coagulation Factor XIa and Uses Thereof,” No. 62/196,037 in a subject to be treated with an FXIa decoy molecule may be lower or higher than the typical therapeutic concentration, for example about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, or about 1,000 nM. These amounts are not meant to be limiting, and increments between the recited amounts are specifically envisioned as part of the disclosure.

In some embodiments, a typical therapeutic dose of an FXIa decoy molecule may be about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1,000 nM, about 1,100 nM, about 1,200 nM, about 1,300 nM, about 1,400 nM, about 1,500 nM, about 1,600 nM, about 1,700 nM, about 1,800 nM, about 1,900 nM, about 2,000 nM, about 5,000 nM, about 7,500 nM, or about 10,000 n114. These amounts are not meant to be limiting, and increments between the recited amounts are specifically envisioned as part of the disclosure.

In some embodiments, an FXIa decoy molecule as described herein is administered at a dose that achieves a plasma concentration of FXIa decoy that is about twice the plasma concentration of the FXIa inhibitor (e.g., an anti-FXIa antibody, e.g., DEF). In some embodiments, a therapeutic plasma concentration for the FXIa inhibitor (e.g., an anti-FXIa antibody, e.g DEF) is from about 40 nM to about 400 nM, about 50 nM to about 300 nM, or about 60 nM to about 200 nM (e.g., about 40 nM about 50 nM about 60 nM, about 70 nM, about 80 nM, about 90 nM, about 100 nM, about 150 nM, about 200 nM, about 250 nM, about 300 nM, about 350 nM, or about 400 nM), and a therapeutic plasma concentration for the FXIa decoy is about 80 nM to about 800 nM, about 100 nM to about 600 nM, or about 120 nM to about 400 nM (e.g., about 80 nM, about 90 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, or about 800 nM).

In some embodiments, an FXIa decoy molecule can be used to counteract a direct FXIa inhibitor in cases of overdose, such as when the plasma concentration of the inhibitor is at least 20% at least 30%, at least 40%, at least 50%, at least 60%, at least 70%, at least 80%, at least 90%, at least 95%, at least 99%, or at least 1.5 fold, at least 2-fold, at least 3-fold, at least 4-fold, at least 5-fold, at least 6-fold, at least 7-fold, at least 10-fold, at least 15-fold at least 20-fold, at least 25-fold, at least 30-fold, or at least 50-fbld higher than the typical therapeutic plasma concentration. These amounts are not meant to be limiting, and increments between the recited amounts are specifically envisioned as part of the disclosure.

The FXIa decoy molecules are effective in counteracting a direct FXIa inhibitor at a plasma concentration that is lower than the plasma concentration of the direct FXIa inhibitor. According to the disclosure, the FXIa decoy molecule counters the effect of a direct FXIa inhibitor at a plasma concentration ratio of inhibitor to decoy of about 1 to 1, about 1 to 5, about 1 to 10, about 1 to 25, about 1 to 50, about I to 100, about 1 to 250, about 1 to 500, about 1 to 1,000, about 1 to 2,500, about 1 to 5,000 or about 1 to 10,000. In certain embodiments, the FXIa decoy molecule counters the effect of a direct FXIa inhibitor at a plasma concentration of at least 10-fold, at least 25-fold, at least 50-fold, at least 100-fold, at least 250-fold, at least 500-fold, at least 1,000-fold, at least 2,500-fold, at least 5,000-fold, or at least 10,000-fold higher than the plasma concentration of the direct FXIa inhibitor. These amounts are not meant to be limiting, and increments between the recited amounts are specifically envisioned as part of the disclosure.

Achieving a target plasma concentration of FXIa decoy molecule sufficient to reverse overdose of a direct FXIa inhibitor is within the knowledge of those ordinarily skilled in the art. In a non-limiting example, estimates of relevant pharmacokinetic parameters, such as subject plasma volume or other parameters, can be made based on upon subject sex, height and weight, or other factors, and used to calculate how much FXIa decoy molecule needs be administered to achieve the target concentration. After administering FXIa decoy molecule, plasma concentrations can be monitored according to the knowledge of those ordinarily skilled in the art and this information used to maintain the concentration in any desired range.

In some embodiments, the compositions and methods of the disclosure use a “therapeutically effective amount” or a “prophylactically effective amount” of an FXIa decoy molecule. A “therapeutically effective amount” refers to an amount effective, at dosages and for periods of time necessary, to achieve the desired therapeutic result. A therapeutically effective amount of the FXIa decoy molecule may vary according to factors such as the disease state, age, sex, and weight of the individual, and the ability of the FXIa decoy molecule to elicit a desired response in the individual. A therapeutically effective amount is also one in which any toxic or detrimental effects of the FXIa decoy molecule are outweighed by the therapeutically beneficial effects. A “prophylactically effective amount” refers to an amount effective, at dosages and for periods of time necessary, to achieve the desired prophylactic result. For example, a dose may be given prior to a planned surgery.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required phamaceutical carrier. The specification for the dosage unit forms of the disclosure are dictated by and directly dependent on (a) the unique characteristics of the FXIa decoy molecule and the particular therapeutic or prophylactic effect to be achieved, and (b) the limitations inherent in the art of compounding such an FXIa decoy molecule for the treatment of individuals.

In certain embodiments, a therapeutically or prophylactically-effective amount of an FXIa decoy molecule administered is about 0.0001 to 50 mg/kg, about 0.001 to 50 mg/kg, about 0.001 to 5 mg/kg, about 0.001 to 0.5 mg/kg, about 0.001 to 005 mg/kg, about 001 to 5 mg/kg or about 0.01 to 0.5 mg/kg. These amounts are not meant to be limiting, and increments between the recited amounts are specifically envisioned as part of the disclosure.

In certain embodiments, a therapeutically or prophylactically-effective serum concentration of an FXIa decoy molecule of the disclosure is about 0.0003 nM to about 3 μM, about 0.003 nM to about 300 nM, about 0.03 nM to about 300 nM, about 0.003 nM to about 30 nM, about 0.03 nM to about 30 nM, about 0.3 nM to about 3 nM, about 0.03 nM to about 3 μM, about 0.3 nM to about 3 μM, about 3 nM to about 3 μM, about 30 nM to about 3 μM, or about 300 nM to about 3 μM. The concentration of the FXIa decoy molecule, for example in blood or plasma, may be measured by any method known in the art. These amounts are not meant to be limiting, and increments between the recited amounts are specifically envisioned as part of the disclosure.

It is to be noted that dosage values may vary with FXIa inhibitor concentration. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The FXIa decoy molecule may be administered, for example in a composition comprising it, once or multiple times to a subject until adequate hemostasis is restored or the direct FXIa inhibitor or inhibitors are no longer effective. Where multiple administrations are used they may administered hourly, daily, or at any other appropriate interval, including for example multiple daily doses. Multiple doses may be administered on a schedule such as every 10 minutes, every 15 minutes, every 20 minutes, every 30 minutes, every hour, every two hours, every three hours, every four hours, three times daily, twice daily, once daily, once every two days, once every three days, and once weekly. The FXIa decoy molecule may also be administered continuously, e.g. via a minipump. The FXIa decoy molecule may be administered, for example, via a parenteral route (e.g., intravenously, subcutaneously, intraperitoneally, or intramuscularly). The FXIa decoy molecule will generally be administered as part of a pharmaceutical composition as described below.

### VII. KITS

In another aspect, kits comprising an FXIa decoy molecule as described herein, or comprising a composition comprising such an FXIa decoy molecule as d.escribed herein, are provided. In addition to the FXIa decoy molecule or composition, a kit may include diagnostic or additional therapeutic agents. A kit can also include instructions for use in a therapeutic method, as well as packaging material such as, but not limited to, ice, dry ice, styrofoam, foam, plastic, cellophane, shrink wrap, bubble wrap, cardboard and starch peanuts.

In one embodiment, the kit includes the FXIa decoy molecule or a composition comprising the FXIa decoy molecule and one or more therapeutic agents that can be used in a method described herein. In some embodiments, a kit comprising an FXIa decoy molecule or a composition comprising the FXIa decoy molecule and one or more additional therapeutic agents is used in a method for decreasing anticoagulant activity in a subject being administered an FXIa inhibitor. In some embodiments, a kit comprising an FXIa decoy molecule or a composition comprising the FXIa decoy molecule and one or more additional therapeutic agents is used in a method for reducing clotting time in a subject being administered an FXIa inhibitor.

### VIII. COMBINATION THERAPIES

In some embodiments, an FXIa decoy molecule as described herein is co-administered with one or more additional therapeutic agents. In some embodiments, co-administration of an FXIa decoy molecule with an additional therapeutic agent (combination therapy) encompasses administering a pharmaceutical composition comprising the FXIa decoy molecule and the additional therapeutic agent, as well as administering two or more separate pharmaceutical compositions, i.e., one comprising the FXIa decoy molecule and the other(s) comprising the additional therapeutic agent(s). In some embodiments, co-administration or combination therapy further includes administering the FXIa decoy molecule and additional therapeutic agent(s) simultaneously or sequentially, or both. For instance, the FXIa, decoy molecule may be administered once every three days, while the additional therapeutic agent is administered once daily at the same as the FXIa decoy molecule, or at a different time. An FXIa decoy molecule may be administered prior to or subsequent to treatment with the additional therapeutic agent. Similarly, administration of an FXIa decoy molecule of the disclosure may be part of a treatment regimen that includes other treatment modalities including surgery. The combination therapy may be administered to prevent recurrence of the condition. The combination therapy may be administered from multiple times hourly to weekly. The administrations may be on a schedule such as every 10 minutes, every 15 minutes, every 20 minutes, every 30 minutes, every hour, every two hours, every three hours, every four hours, three times daily, twice daily, once daily, once every two days, once every three days, once weekly, or may be administered continuously, e.g. via a minipump. The combination therapy may be administered, for example, via a parenteral route (e.g., intravenously, subcutaneously, intraperitoneally, or intramuscularly).

In some embodiments, the additional therapeutic agents are lipid lowering compounds; compounds suitable for the treatment of coronary diseases and/or compounds exhibiting vasodilatative activities; diuretics; inhibitors of calcium channels; inhibitors of the coagulation cascade; and anticoagulants like non-fractionated heparins, low molecular weight heparins, hirudin, bivalirudin and/or argatroban. Examples of suitable combination therapeutics are provided in WO2013167669, incorporated herein by reference.

In another embodiment, the FXIa decoy molecule may be co-administered with another procoagulant including another FXIa decoy molecule, Factor IX, Factor Xa, Factor XIIa, Factor VIII, Factor VIIa, FEMA, fresh frozen plasma, a Factor Xa inhibitor (e.g., andexanet alfa), and prothrombin complex concentrate (PCC), and/or a fibrinolysis inhibitor (e.g., epsilon aminocaproic acid (EACA) or tranaxemic acid (TXA)).

### IX. COMPOSITIONS

In a further aspect, the disclosure provides a composition comprising an FXIa decoy molecule for use in counteracting a direct FXIa inhibitor in a subject. The composition may comprise a pharmaceutically acceptable carrier, vehicle or other ingredients that are physiologically compatible. Non-limiting examples of such carriers, vehicles and other ingredients include solvents (e.g., water, ethanol, saline, phosphate buffered saline), detergents, surfactants, dispersion media, coatings, antibacterial or antifungal agents, isotonifying agents, absorption delaying agents, sugars (e.g., sucrose, dextrose, lactose), polyalcohols (e.g., glycerol, mannitol, sorbitol), salts (e.g., sodium chloride, potassium chloride), wetting agents, emulsifying agents, preservatives, buffers, and agents capable of enhancing the stability or effectiveness of the FXIa decoy molecule.

A composition for use according to the disclosure may be in any suitable form for administration to a subject, such as liquid solutions (e.g., injectable and infusible solutions). Compositions can be provided in a pre-mixed format ready for administration to a subject, for example, in a vial or pre-filled syringe. Such formats do not require reconstitution with diluent before administration. Alternatively, compositions can be provided in lyophilized form requiring reconstitution with diluent (e.g., sterile water or saline) before administration. If the latter, diluent can be provided with the lyophilisate in a separate container. According to the knowledge of those of ordinary skill in the art, compositions can be formulated for storage under refrigeration or at room temperature. The form of the composition depends, at least in part, on the intended mode of administration. In certain embodiments, the mode of administration is parenteral, including for example intravenous, subcutaneous, intraperitoneal, or intramuscular administration.

Therapeutic compositions typically are sterile and stable under the conditions of manufacture and storage. The composition can be formulated as a solution, microemuision, dispersion, in liposomes, or other ordered structure suitable to high drug concentration. Sterile injectable solutions can be prepared by incorporating the FXIa decoy molecule in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

It is further contemplated by the present disclosure that any of the compositions herein may be administered to a subject being treated with a direct FXIa inhibitor (e.g., an anti-FXIa antibody).

It is understood that the examples and embodiments described herein are for illustrative purposes only and that various modifications or changes in light thereof will be apparent to persons skilled in the art and are to be included within the and can be made without departing from the true scope of the disclosure.

### X. EXAMPLES

The following examples are provided to illustrate, but not to limit, the claimed invention.

### Example 1

### Production and Purification of FXIa Catalytic Domains

Experiments were conducted to generate an FXIa-derivative lacking procoagulant function but capable of binding to active site-directed FXIa-specific antibodies and small molecule FXIa active site inhibitors. The catalytic domain of human FXI (i.e., IIe370 to Ala606 of SEQ ID NO:1) was expressed in human embryonic kidney (HEK) 293 cells. Expression plasmids encoded a mammalian signal peptide derived from mouse IgG (SEQ ID NO:4), followed by the catalytic domain of human FXI and a octa-histidine tag (His₈) at the C-terminus to facilitate detection and/or purification of the protein. The catalytically active construct contained a Cys482Ser substitution and the catalytically inactive variant (FXI _{S557A}) Cys482Ser and Ser557Ala substitutions see, e.g., Aktimur et al., 2003,1 J. Biol. Chem. 278(10):7981-7987; Navaneetham et al, 2005, J. Biol. Chem. 280(4):36165-36175). Suspension HEK 293 cell cultures were transiently transfected using lipofection and conditioned media was harvested 4 days post transfection. Cells were pelleted by centrifugation and the protein was purified from the supernatant using nickel sepharose resin per the manufacturer's instructions (GE Healthcare). Captured protein was eluted using imidazole buffer and the buffer was exchanged via dialysis into phosphate buffer saline (PBS). The purity and size of the protein was analyzed by SDS-PAGE and the protein concentration measured using the absorbance at 280 nm.

The interaction between the FXI catalytic domain and DEF IgG was confirmed using a Biacore T200 Instrument (GE Healthcare). DEF IgG was captured using an anti-Human IgG antibody immobilized on a Biacore CMS chip using the Human Antibody Capture Kit (GE Healthcare). Purified catalytically inactive protein comprising the double substitution C482S and S557A, hereinafter referred to as “FXI_{S557A},” was diluted to 20 nM in the running buffer containing 10 mM HEPES (pH7.4), 150 mM NaCl and 0.005% v/v Surfactant P20 (HBSp) buffer (GE Healthcare), injected over the immobilized DEF IgG for 180 sec, and allowed to dissociate for an additional 180 sec with a flow rate of 30 μl/min at 25° C. Data was background subtracted using the signal from the adjacent flow cell with only anti-Human IgG immobilized and no DEF IgG captured.

Enzyme activity was determined using a fluorogenic peptide substrate cleavage assay. Reactions were initiated by mixing blood-derived FXIa (0.11 μg/ml) (Haematologic Technologies Inc.) with100 μM SN-59 peptide substrate (Haematologic Technologies Inc) in assay buffer containing 50 mM Tris-HCl (pH7.4), 250 mM NaCI and 1 mM EDTA. To confirm the activity of the catalytic domains, 50 nM purified FXI catalytic domain was mixed with the SN-59 peptide in assay buffer. The reaction was monitored in a SpectraMax fluorescent plate reader (Molecular Devices) at 37° C. with an excitation of 352 nm and emission of 370 nm. Data was collected every minute and results reported as the Vₘₐₓ (units/sec) for 50-1000 sec.

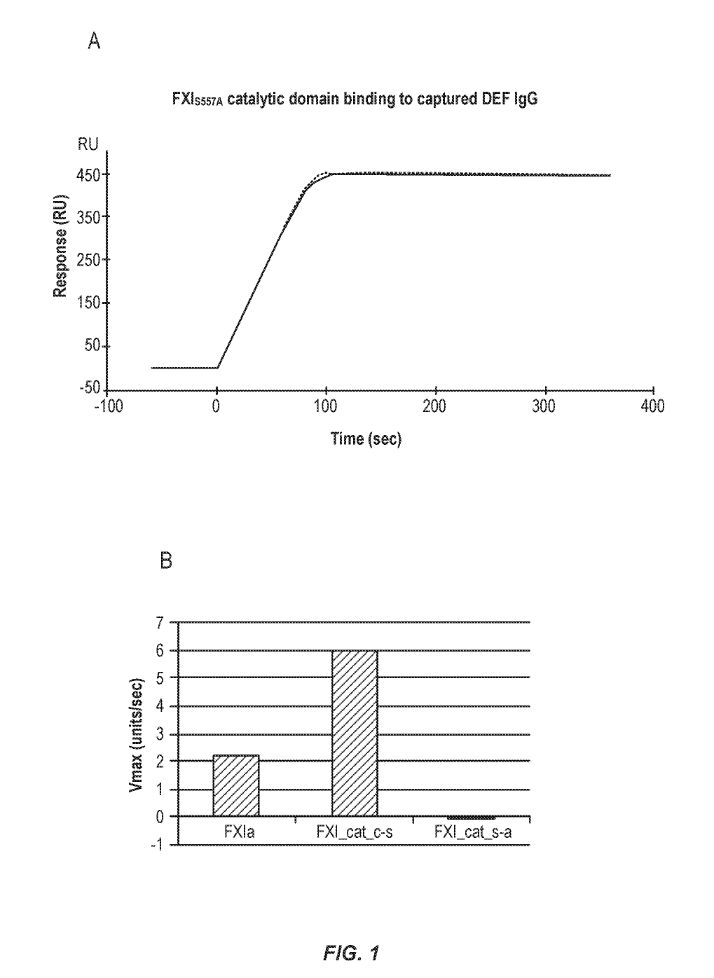

Results: Purified FXI_{S557A} catalytic domain maintained binding affinity for DEF IgG and exhibited a slow off-rate (FIG. 1A). The isolated FXI catalytic domain maintained enzyme activity (FIG. 1B, FXI_cat_c-s). No activity was detected at an equivalent concentration of the FXI_{S557A}-substituted catalytic domain (FIG. 1B, FXI_cat_s-a). Thus, FXI_{S557A} bound an anti-FXIa antibody but was catalytically inactive (i.e., did not cleave a known FXIa substrate).

These results indicated that the FXI_{S557A} catalytic domain decoy molecule lacked catalytic activity but also maintained the relevant wild-type conformation so the DEF-binding epitope could still be recognized by the antibody.

### Example 2

### Ability of the FXI_{S557A} Catalytic Domain to Interfere with the Inhibitory Activity of DEF IgG in an In Vitro Enzyme Activity Assay

The ability for the inactive FXI_{S557A} catalytic domain to inhibit DEF IgG antagonist activity was assessed by premixing blood-derived FXIa (0.11 μg/ml; about 0.7 nM) and FXI_{S557A} catalytic domain (10, 20, 100 and 500 nM) before adding the DEF antibody (10 nM) for 5 minutes and initiating the reaction with the peptide substrate SN-59 substrate (Haematologic Technologies Inc) in assay buffer containing 50 mM Tris-HCl (pH 7.4), 250 mM NaCl and 1 mM EDTA. The reaction was monitored using a SpectraMax fluorescent plate reader (Molecular Devices) at 37° C. with an excitation of 352 nm and emission of 370 nm. Data was collected every minute and results were reported at the V ₘₐₓ (units/sec) for 50-1000 sec.

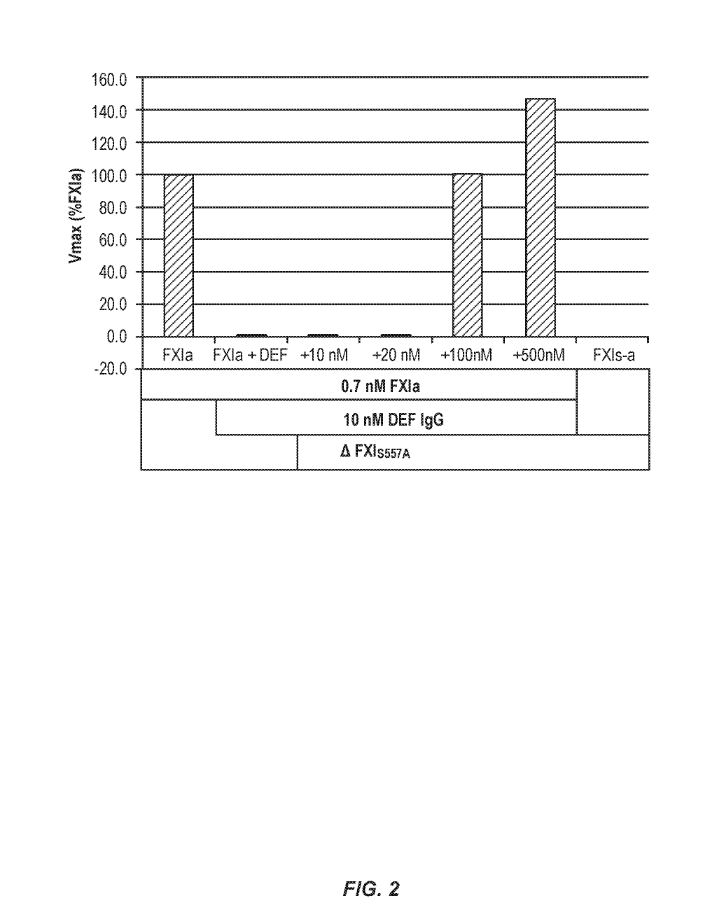

Results: 10 nM DEF completely inhibited the enzyme activity of FXIa in the fluorogenic peptide substrate assay (FIG. 2, FXIa+DEF). Addition of FXI_{S557A} catalytic domain prevented such inhibition (FIG. 2, +100 nM, +500 nM). A high concentration of FXI_{S557A} (500 nM) in the absence of FXIa showed no catalytic activity (FIG. 2. FXIs-a) This result showed that, in the absence of an FXIa inhibitor, the decoy did not alter or contribute FXIa activity, but did, in the context of the activity of an anti-FXIa mAb (e.g., DEF), reverse the effects of an FXIa-specific inhibitor. This suggested that the FXI_{S557A} may be a potential novel “reversal agent” therapeutic in the context of treatment with FXIa inhibitors, such as, but not limited to, a reversal agent for an anti-FXIa mAb DEF). In some cases, FXIa inhibitors may result in unwanted side effects, such as excess bleeding, due to unanticipated sensitivity of individual patients, unscheduled surgery or trauma—relatively common occurrences in present day anticoagulant therapy. Thus, FXI_{S557A} may provide a novel therapeutic to reverse any unwanted or deleterious effect of FXIa inhibitor therapy.

### Example 3

### Binding Affinity of the FXI_{S557A} Catalytic Domain for DEF Fab

Binding affinity was measured using a Biacore T200 Instrument (GE Healthcare). Recombinant FXI_{S557A} catalytic domain was biotin labeled via primary amines and captured on a Biacore CAP chip (GE Healthcare). Experiments were performed at 37° C. using a 30 μ/min flow rate in 10 mM HEPES (pH 7.4), 150 mM NaCI and 0.005% v/v Surfactant P20 (HBSp) buffer (GE Healthcare). After each DEF Fab injection, the chip surface was regenerated with a solution of 6 M guanidine-HCl and 0.25 M NaOH, coated with the CAP reagent and new FXI_{S557A} was captured. All data was analyzed using the Biacore T200 Evaluation software and background and buffer subtracted data fit to a 1:1 Langmuir binding model. Results were reported as the mean of two experiments.

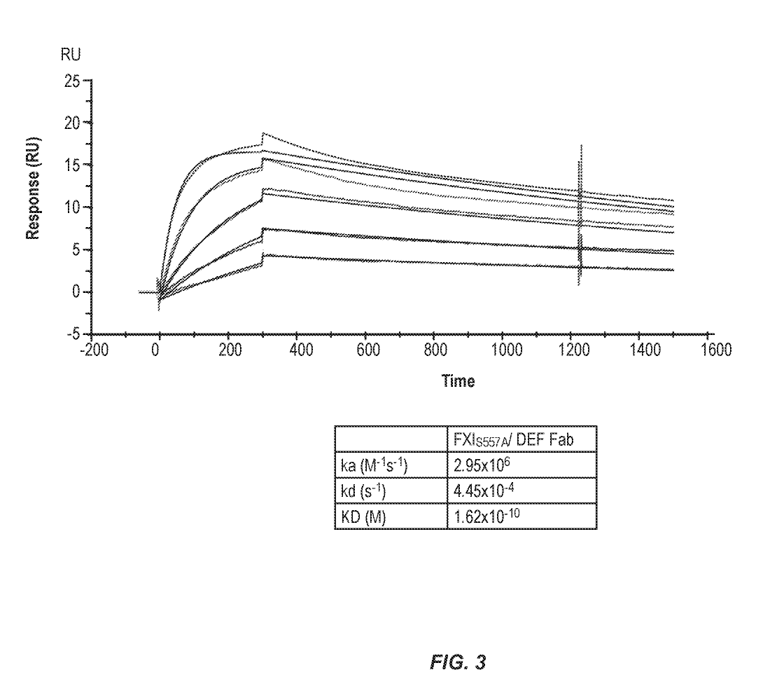

Results: The FXI_{S557A} catalytic domain binds to DEF Fab at 37° C. with an affinity (K_{D}) of 162 pM (FIG. 3). This result showed that the FXI_{S557A} decoy has high affinity for the anti-FXIa mAb DEF and that a reasonable dosing protocol, for any future therapeutic applications, is likely achievable.

### Example 4

### Reversal Effects of FXI_{S557} Catalytic Domain Decoy on Different Anti-FXIa mAbs

The ability of the inactive FXI_{S557A} catalytic domain to inhibit the activity of different anti-FXIa antibodies with different affinities for FXIa, and different CDR sequences, was assessed by premixing the individual antibodies (DEF, DEW, B10B12, and D4, each of which is further described in U.S. Provisional Patent Application “Antibodies to Coagulation Factor XIa and Uses Thereof,” No. 62/196,037) with the FXI_{S557A} catalytic domain (FXIs_{S557A} catalytic domain in a fixed 3:1 ratio to IgG over the IgG dose range of 25.6, 8.6. 2.85, 0.95, 0.31, 0.1, 0.35 μg/ml) prior to adding human FXIa (300 pM) and SN-59 substrate (100 μM; Haematologic Technologies Inc) under normal assay buffer conditions. The reaction was monitored in a SpectraMax fluorescent plate reader (Molecular Devices) at 37° C. with an excitation of 352 nm and emission of 370 nm. Data was collected every minute and results reported at the Vₘₐₓ (units/sec) for 50-1000 sec.

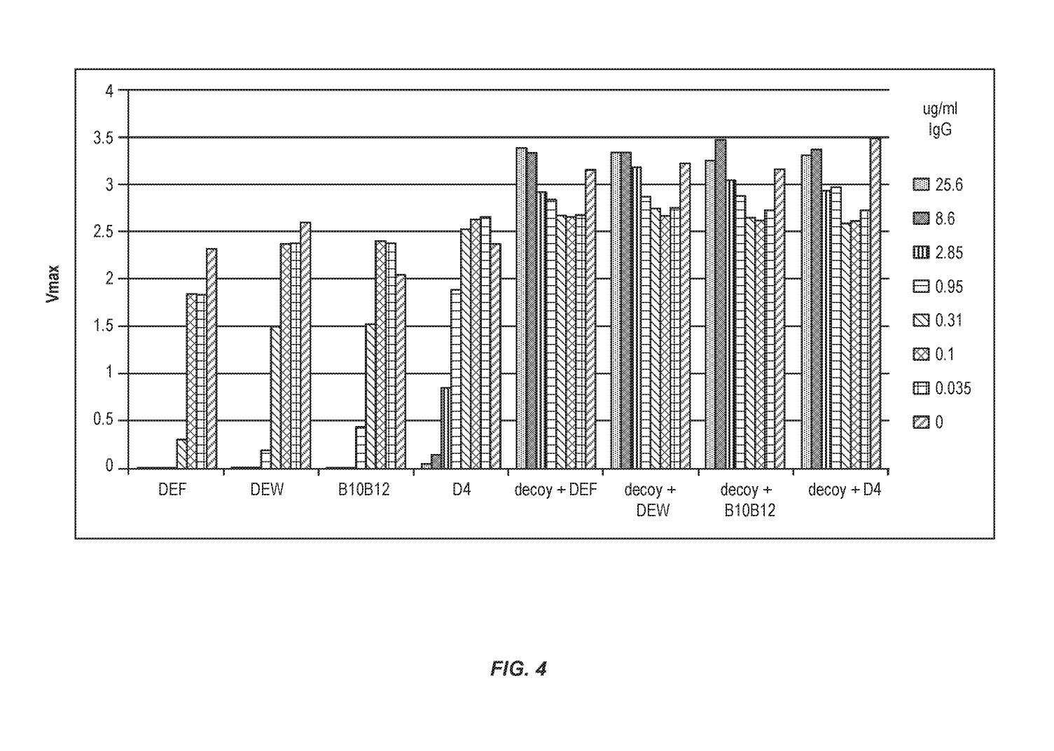

Results: In this example, the FXI_{S557A} catalytic domain decoy was pre-mixed in a 3:1 ratio with different anti-FXIa mAbs that inhibit FXIa activity. These mAbs have different CDR sequences and different potencies in inhibiting FXIa activity. In all cases shown, the FXI_{S557A} catalytic domain decoy was able to reverse the inhibitory effects of the anti-FXIa mAbs on FXIa activity (FIG. 4).

Without wishing to be bound by any theory, these data suggest that the FXI_{S557A} catalytic domain polypeptide is a potential useful therapeutic to reverse the effect of a various large molecule, e.g., antibody, FXIa inhibitors since the reversal effect was observed with different anti-FXIa inhibitor antibodies with different sequences and binding characteristics. It also raises the possibility that the FXI_{S557A} inactive catalytic domain polypeptide may be able to reverse the effects of other agents, including small molecule inhibitors, which bind to the FXIa catalytic domain.

### Example 5

### Reversal Effects of FXI_{S557A} Catalytic Domain Decoy on Inhibition of FXIIa-Triggered Thrombin Generation by DEF in Human Plasma

Thrombin generation was measured using a fluorogenic thrombin substrate on a multi-well automated fluorescent plate reader (ThrombinoSCOPE, Maastricht, the Netherlands) according to the manufacturer's protocol. Briefly, 5 μL, of anti-FXIa DEF antibody (16 μg/ml) was added in combination with different ratios of the FXI_{S557A} catalytic domain decoy molecule (3.1, 6.3, 12.5, 25, 50, 100 μg/ml) and mixed with 20 μL PBS-60 nM human Factor XIIa (Enzyme Research Laboratories, South Bend, Ind., USA) and PC/PS (Phospholipid-TGT, DiaPharma., West Chester, Ohio, USA) in a 96-well plate, Finally, 75 μL human plasma (Triclinical Reference Plasma, TCoag, Wicklow, Ireland) was added. Due to lot-to-lot variability for the PC/PS reagent, for each lot the concentration was adjusted to achieve a ˜10 min Lag Time and ˜125 nM Thrombin Peak in control plasma. Finally, thrombin generation was triggered and measured by the addition of calcium chloride buffer and a fluorogenic thrombin substrate. The amount of thrombin generated in the reaction was measured over time.

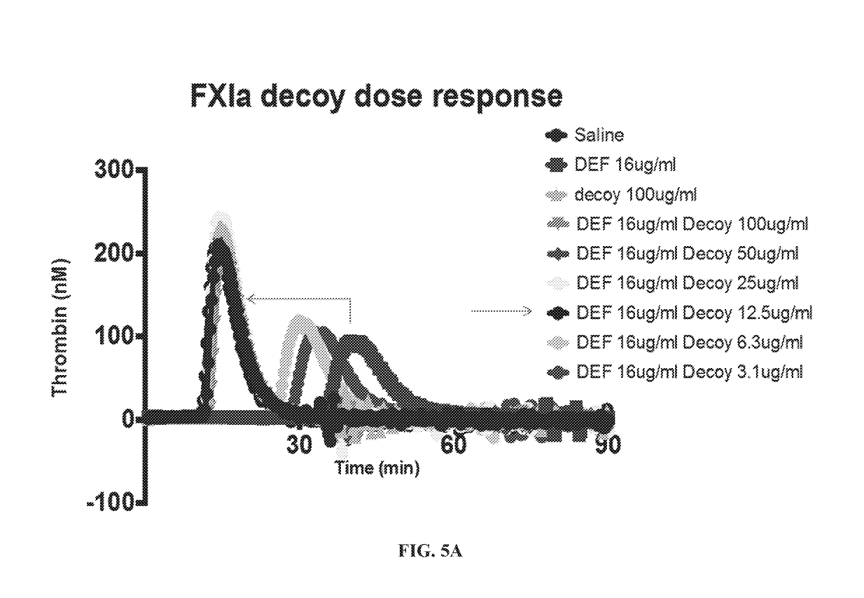

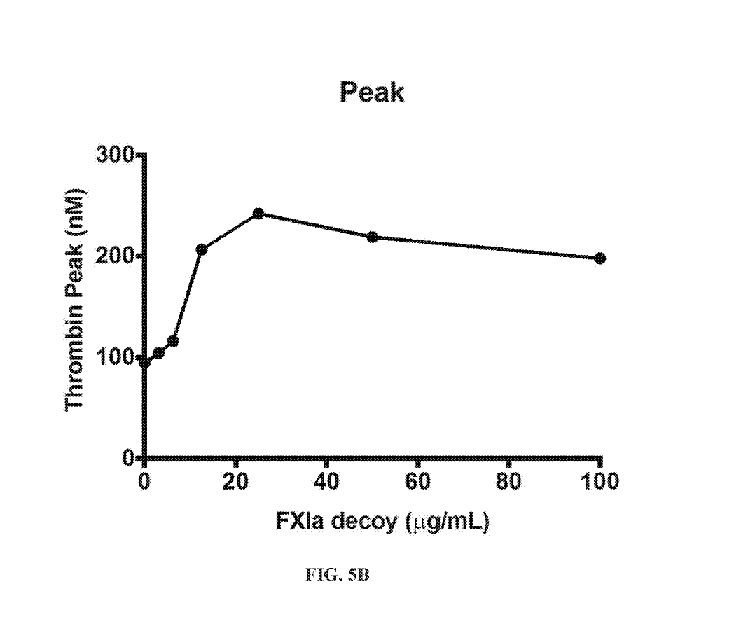

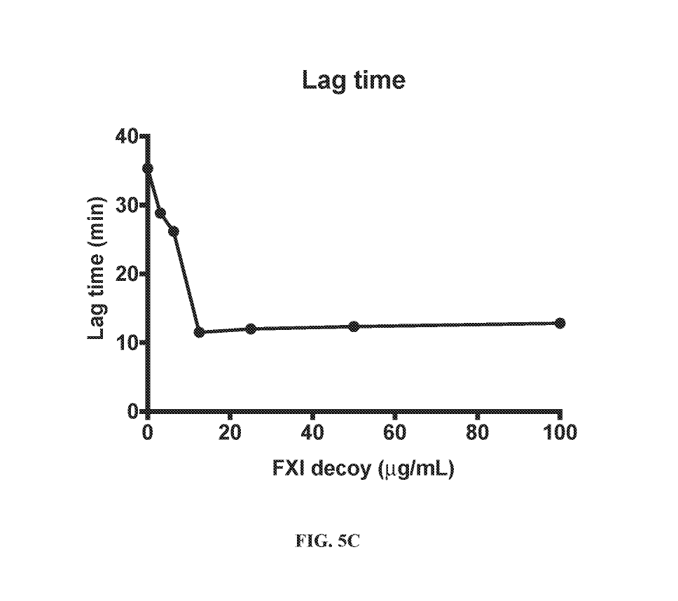

Results: The FXI_{S557A} catalytic domain decoy molecule reversed the inhibitory effects of the anti-FXIa mAb DEF on FXIIa-triggered thrombin generation in a human plasma assay (FIGS. 5A, B, and C). Reversal of DEF effects was seen for all doses at or above 12.5 μg/ml of the decoy, indicating that the reversal properties were first evident in this assay at an ˜5:1 ratio of decoy to DEF mAb (or, since each mAb has two binding sites, an approximately 2-fold ratio of decoy to binding sites).

These results show that the FXI_{S557A} catalytic domain decoy molecule itself had no effect on the FXIIa-triggered coagulation process in human plasma but did reverse the inhibitory effect of DEF on this process. Without wishing to be bound by any particular theory, confirmation that the decoy functioned as predicted in human plasma and at a ratio of decoy to antibody that was practical supports the possibility that FXI_{S557A} is a potential therapeutic to reverse the effects of an FXIa inhibitor.

### Example 6

### FXI_{S574} Catalytic Domain Decoy Reverses Effects of Anti-FXia Mab DEF in an In Vivo Rabbit Dosing Experiment

All procedures performed on these animals were in accordance with regulations and established guidelines and were reviewed and approved by an Institutional Animal Care and Use Committee or through an ethical review process. Rabbits were anesthetized according to established protocols. A 90 minute in life rabbit study was then carried out involving the following procedures. Four rabbits were treated, with all animals being treated the same. At time 0 each animal received a 1 mg/kg bolus injection of the DEF IgG, 30 minutes later each animal then received a bolus injection of 1 mg/kg decoy. Just prior to each bolus injection, and 30 minutes after the final (decoy) injection, blood was drawn to make plasma for APTT and PT clotting time assays. Data is plotted to show the sequential effects of DEF IgG and decoy injection on APTT and PT coagulation times.

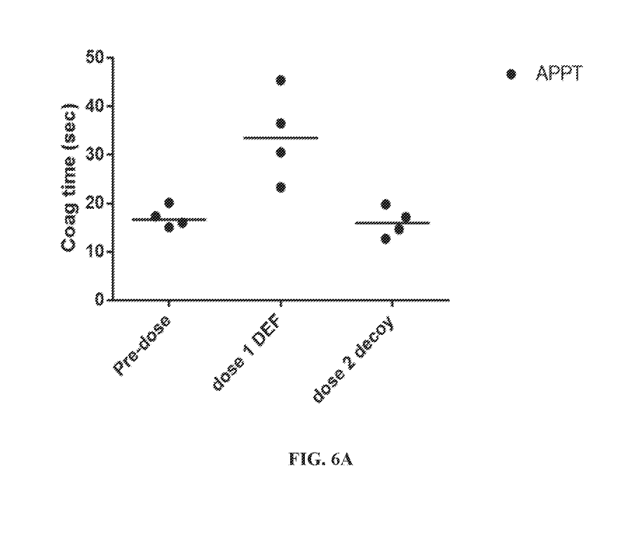

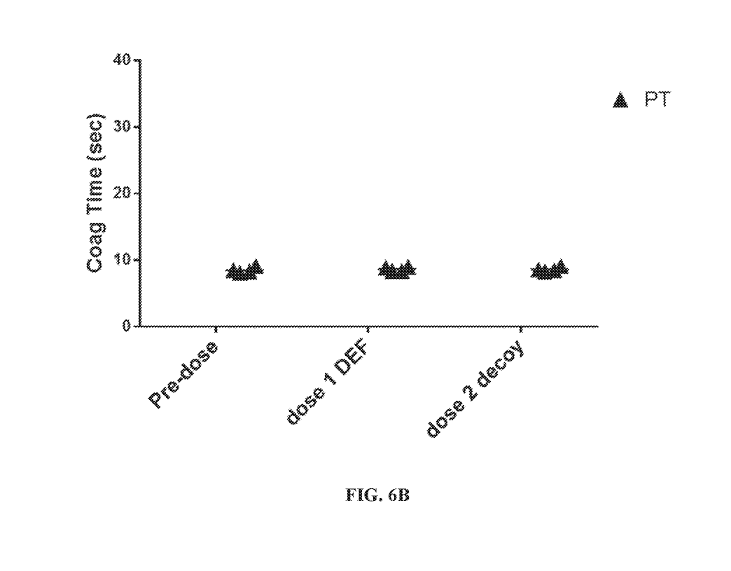

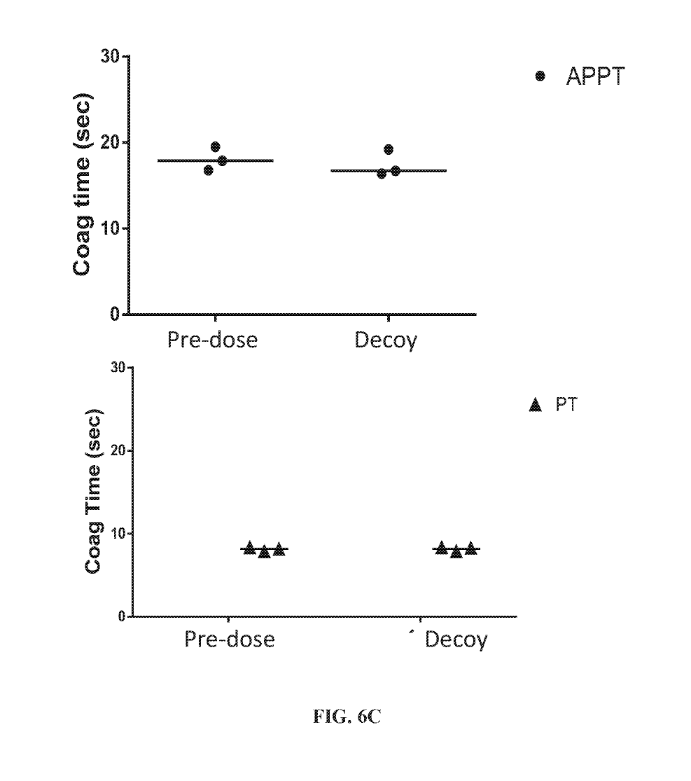

Results: The results of this sequential dosing experiment in the live rabbit show that the FXI_{S557A} catalytic domain decoy molecule reverses the effects of the DEF mAb, as measured in the ex vivo APTT assay, 30 minutes after dosing with the decoy molecule (FIG. 6A). This result provides further evidence that dosing of the decoy could quickly reverse the effects of DEF dosing in vivo were any DEF-related adverse bleeding events to occur. No effects on PT coagulation times were seen between pre- and post-dose samples, as expected (FIG. 6B). Control animals dosed with decoy alone (i.e., no dose of DEF) did not exhibit changes in coagulation in either an APTT or PT Assay (FIG. 6C).

TABLE 2
Sequences

| | | |
|---|---|---|
| Full length Factor XIa | SEQ ID | ECVTQLLKDTCFEGGDITTVFTPSAKYCQVVCTYHPRC |
| protein sequence | NO: 1 | LLFTFTAESPSEDPTRWETCVLKDSVTETLPRVNRTAAI |
| mature peptide) | | SGYSFKQCSHQISACNKDIYVDLDMKGINYNSSVAKS |
| | | AQECQERCTDDVHCHFFTYATRQFPSLEHRNICLLKHT |
| | | QTGTPTRITKLDKVVSGFSLKSCALSNLACIRDIFPNTV |
| | | FADSNIDSVMAPDAFVCGRICTHHPGCLFFTFFSQEWP |
| | | KESQRNLCLLKTSESGLPSTRIKKSKALSGFSLQSCRHS |
| | | IPVFCHSSFYHDTDFLGEELDIVAAKSHEACQKLCTNA |
| | | VRCQFFTYTPAQASCNEGKGKCYLKLSSNGSPTKILHG |
| | | RGGISGYTLRLCKMDNECTTKIKPRIVGGTASVRGEWP |
| | | WQVTLHTTSPTQRHLCGGSIIGNQWILTAAHCFYGVES |
| | | PKILRVYSGILNQSEIKEDTSFFGVQEIIIHDQYKMAESG |
| | | YDIALLKLEYYVNYTDSQRPICLPSKGDRNVIYTDCWV |
| | | TGWGYRKLRDKIQNTLQKAKIPLVTNEECQKRYRGH |
| | | KITHKMICAGYREGGKDACKGDSGGPLSCKHNEVWH |
| | | LVGITSWGEGCAQRERPGVYTNVVEYVDWILEKTQA |
| | | V |
| FXI_{S557A} catalytic | SEQ ID | IVGGTASVRGEWPWQQVTLHTTSPTQRHLCGGSIIGNQ |
| domain mature protein | NO: 2 | WILTAAHCFYGVESPKILRVYSGILNQSEIKEDTSFFGV |
| sequence (mutated | | QEIIIHDQYKMAESGYDIALLKLETTVNYTDSQRPISLP |
| residues at positions | | SKGDRNVIYTDCWVTGWGYRKLRDKIQNTLQKAKIPL |
| 557 and 482 are | | VTNEECQKRYRGHKITHKMICAGYREGGKDACKGDA |
| bolded and underlined) | | GGPLSCKHNEVWHLVGITSWGEGCAQRERPGVYTNV |
| | | VEYVDWILEKTQA |
| Nucleotide sequence | SEQ ID | ATCGTTGGAGGAACTGCGTCTGTTCGTGGTGAGTGG |
| encoding FXI_{S557A} | NO: 3 | CCGTGGCAGGTGACCCTGCACACAACCTCACCCACT |
| catalytic domain | | CAGAGACACCTGTGTGGAGGCTCCATCATTGGAAAC |
| mature protein | | CAGTGGATATTAACAGCCGCTCACTGTTTCTATGGG |
| sequence | | GTAGAGTCACCTAAGATTTTGCGTGTCTACAGTGGC |
| | | ATTTTAAATCAATCTGAAATAAAAGAGGACACATCT |
| | | TTCTTTGGGGTTCAAGAAATAATAATCCATGATCAG |
| | | TATAAAATGGCAGAAAGCGGGTATGATATTGCCTTG |
| | | TTGAAACTGGAAACCACAGTGAATTACACAGATTCT |
| | | CAACGACCCATAAGCCTGCCTTCCAAAGGAGATAG |
| | | AAATGTAATATACACTGATTGCTGGGTGACTGGATG |
| | | GGGGTACAGAAAACTAAGAGACAAAATACAAAATA |
| | | CTCTCCAGAAAGCCAAGATACCCTTAGTGACCAACG |
| | | AAGAGTGCCAGAAGAGATACAGAGGACATAAAATA |
| | | ACCCATAAGATGATCTGTGCCGGCTACAGGGAAGG |
| | | AGGGAAGGACGCTTGCAAGGGAGATGCGGGAGGCC |
| | | CTCTGTCCTGCAAACACAATGAGGTCTGGCATCTGG |
| | | TAGGCATCACGAGCTGGGGCGAAGGCTGTGCTCAA |
| | | AGGGAGCGGCCAGGTGTTTACACCAACGTGGTCGA |
| | | GTACGTGGACTGGATTCTGGAGAAAACTCAAGCA |
| Signal peptide amino | SEQ ID | MGWSCIILFLVATATGVHS |
| acid sequence | NO: 4 | |
| Signal peptide | SEQ ID | ATGGGCTGGTCCTGTATCATCCTCATTTCTGGTGGCCA |
| nucleotide sequence NO: 5 | | CAGCTACCGGAGTGCATAGC |
| FXI_{S557A} catalytic | SEQ ID | mgwsciilflvatatgvhs/IVGGTASVRGEWPWQVTLHTTSPT |
| domain protein | NO: 6 | QRHLCGGSIIGNQWILTAAHCFYGVESPKILRVYSGILN |
| sequence with signal | | QSEIKEDTSFFGVQEIIIHDQYKMAESGYDIALLKLETT |
| peptide and His tag | | VNYTDSQRPISLPSKGDRNVIYTDCWVTGWGYRKLRD |
| mutated residues at | | KIQNTLQKAKIPLVTNEECQKRYRGHKITHKMICAGY |
| positions 557 and 482 | | REGGKDACKGDAGGPLSCKHNEVWFLVGITSWGEGC |
| are bolded and | | AQRERPGVYTNVVEYVDWILEKTQAHHHHHHHH |
| underlined; / indicates | | |
| signal peptidase | | |
| cleavage site) | | |
| FXI_{S557A} catalytic | SEQ ID | mgwsciilflvatatgvhs/IVGGTASVRGEWPWQVTLHTTSPT |
| domain protein | NO: 7 | QRHLCGGSIIGNQWILTAAHCFYGVESPKILRVYSGILN |
| sequence with signal | | QSEIKEDTSFFGVQEIIIHDQYKMAESGYDIALLKLETT |
| peptide (mutated | | VNYTDSQRPISLPSKGDRNVIYTDCWVTGWGYRKLRD |
| residues at positions | | KIQNTLQKAKIPLVTNEECQKRYRGHKITHKMICAGY |
| 557 and 482 are | | REGGKDACKGDAGGPLSCKHNEVWHLVGITSWGEGC |
| bolded and underlined; | | AQRERPGVYTNVVEYVDWILEKTQA |
| / indicates signal | | |
| peptidase cleavage | | |
| site) | | |
| Nucleotide sequence | SEQ ID | ATGGGCTGGTCCTCTTATCATCCTCTTTCTGGTGGCCA |
| encoding FX_{S557A} | NO: 8 | CAGCTACCGGAGTGCATAGCATCGTTGGAGGAACTG |
| catalytic domain | | cGTcTGTTCGTGGTGAGTGGCCGTGGCAGGTGACCC |
| mature protein | | TGCACACAACCTCACCCACTCAGAGACACCTGTGTG |
| sequence and an N- | | GAGGCTCCATCATTGGAAACCAGTGGATATTAACAG |
| terminal signal peptide | | CCGCTCACTGTTTCTATGGGGTAGAGTCACCTAAGA |
| of SEQ ID NO: 4. | | TTTTGCGTGTCTACAGTGGCATTTTAAATCAATCTGA |
| | | AATAAAAGAGGACACATCTTTCTTTTGGGGTTCAAGA |
| | | AATAATAATCCATGATCAGTATAAAATGGCAGAAA |
| | | GCGGGTATGATATTGCCTTGTTGAAACTGGAAACCA |
| | | CAGTGAATTACACAGATTCTCAACGACCCATAAGCC |
| | | TGCCTTCCAAAGGAGATAGAAATGTAATATACACTG |
| | | ATTGCTGGGTGACTGGATGGGGGTACAGAAAACTA |
| | | AGAGACAAAATACAAAATACTCTCCAGAAAGCCAA |
| | | GATACCCTTAGTGACCAACGAAGAGTGCCAGAAGA |
| | | GATACAGAGGACATAAAATAACCCATAAGATGATC |
| | | TGTGCCGGCTACAGGGAAGGAGGGAAGGACGCTTG |
| | | CAAGGGAGATGCGGGAGGCCCTCTGTCCTGCAAAC |
| | | ACAATGAGGTCTGGCATCTGGTAGGCATCACGAGCT |
| | | GGGGCGAAGGCTGTGCTCAAAGGGAGCGGCCAGGT |
| | | GTTTACACCAACGTGGTCGAGTACGTGGACTGGATT |
| | | CTGGAGAAAACTCAAGCA |
| DEF antibody VH | SEQ ID | EVQLVQSGAEVKKPGASVKVSCKASGYTFTGYYMHW |
| sequence | NO: 9 | VRQAPGQGLEWMGWIDPDEGDTNYAQKFQGRVTMT |
| | | RDTSISTAYMELSRLRSDDTAVYYCARLASGFRDYWG |
| | | QGTLVTVSS |
| DEF antibody VL | SEQ ID | DIVMTKSPSSLSASVGDRVTITCRASQGIRNDLGWYQQ |
| sequence | NO: 10 | KPGKAPKRLIYAASSLQSGVPSRFSGSGSGTEFTLTISSL |
| | | QPEDFATYYCLQHDIYASTFGPGTKVDIKR |
| Full length FXI_{S557A} | SEQ ID | ECVTQLLKDICFEGGDITTVFTPSAKYCQVVCTYHPRC |
| protein sequence | NO: 11 | LLFTFTAESPSEDPTRWFTCVLKDSVTETLPRVNRTAAI |
| (mutated residues at | | SGYSFKQCSHQISACNKDIYVDLDMKGINYNSSVAKS |
| position 557 S → A) | | AQECQERCTDDVHCHFFTYATRQFPSLEHRNICLLKHT |
| | | QTGTPTRITKLDKVVSGFSLKSCALSNLACIRDIFPNTV |
| | | FADSNIDSVMAPDAFVCGRICTHHPGCLFFTFFSQEWP |
| | | KESQRNLCLLKTSESGLPSTRIKKSKALSGFSLQSCRHS |
| | | IPVFCHSSFYHDTDFLGEELDIVAAKSHEACQKLCTNA |
| | | VRCQFFTYTPAQASCNEGKGKCYLKLSSNGSPTKILHG |
| | | RGGISGYTLRLCKMDNECTTKIKPRIVGGTASVRGEWP |
| | | WQVTLHTTSPTQRHLCGGSIIGNQWILTAAHCFYGVES |
| | | PKILRVYSGILNQSEIKEDTSFFGVQEIIIHDQYKMAESG |
| | | YDIALLKLETTVNYTDSQRPICLPSKGDRNVIYTDCWV |
| | | TGWGYRKLRDKIQNTLQKAKIPLVTNEECQKRYRGH |
| | | KITHKMICAGYREGGKDACKGDAGGPLSCKHNEVWH |
| | | LVGITSWGEGCAQRERPGVYTNVVEYVDWILEKTQA |
| | | V |
| Full length FXI_{S557A} | SEQ ID | ECVTQLLKDTCFEGGDITTVFTPSAKYCQVVCTYHPRC |
| protein sequence | NO: 12 | LLFTFTAESPSEDPTRWFTCVLKDSVTETLPRVNRTAAI |
| (mutated residues at | | SGYSFKQCSHQISACNKDFYVDLDMKGINYNSSVAKS |
| position 482 C → 5) | | AQECQERCTDDVHCHFFTYATRQFPSLEHRNICLLKHT |
| | | QTGTPTRITKLDKVVSGFSLKSCALSNLACIRDIFPNTV |
| | | FADSNIDSVMAPDAFVCGRICTHHPGCLFFTFFSQEWP |
| | | KESQRNLCLLKTSESGLPSTRIKKSKALSGFSLQSCRHS |
| | | IPVFCHSSFYHDTDFLGEELDIVAAKSHEACQKLCTNA |
| | | VRCQFFTYTPAQASCNEGKGKCYLKLSSNGSPTKILHG |
| | | RGGISGYTLRLCKMDNECTTKIKPRIVGGTASVRGEWP |
| | | WQVTLHTTSPTQRHLCGGSIIGNQWILTAAHCFYGVES |
| | | PKILRVYSGILNQSEIKEDTSFFGVQEIIIHDQYKMAESG |
| | | YDIALLKLETTVNYTDSQRPISLPSKGDRNVIYTDCWV |
| | | TGWGYRKLRDKIQNTLQKAKIPLVTNEECQKRYRGH |
| | | KITHKMICAGYREGGKDACKGDSGGPLSCKHNEVWH |
| | | LVGITSWGEGCAQRERPGVYTNVVEYVDWILEKTQA |
| | | V |
| Full length FXI_{S557A} | SEQ ID | ECVTQLLKDTCFEGGDITTVFTPSAKYCQVVCTYHPRC |
| protein sequence | NO: 13 | LLFTFTAESPSEDPTRWFTCVLKDSVTETLPRVNRTAAI |
| (mutated residues at | | SGYSFKQCSHQISACNKDIYNDLDMKGINYNSSVAKS |
| positions 557 S → A | | AQECQERCTDDVHCHFFTYATRQFPSLEHRNICLLKHT |
| and 482 C → 5) | | QTGTPTRITKLDKVVSGFSLKSCALSNLACIRDIFPNTV |
| | | FADSNIDSVMAPDAFVCGRICTHHPGCLFFTFFSQEWP |
| | | KESQRNLCLLKTSESGLPSTRIKKSKALSGFSLQSCRHS |
| | | IPVFCHSSFYHDTDFLGEELDIVAAKSHEACQKLCTNA |
| | | VRCQFFTYTPAQASCNEGKGKCYLKLSSNGSPTKILHG |
| | | RGGISGYTLRLCKMDNECTTKIKPRIVGGTASVRGEWP |
| | | WQVTLHTTSPTQRHLCGGSIIGNQWILTAAHCFYGVES |
| | | PKILRVYSGILNQSEIKEDTSFFGVQEIIIHDQYKMAESG |
| | | YDIALLKLETTVNYTDSQRPISLPSKGDRNVIYTDCWV |
| | | TGWGYRKLRDKIQNTLQKAKIPLVTNEECQKRYRGH |
| | | KITHKMICAGYREGGKDACKGDAGGPLSCKHNEVWH |
| | | LVGITSWGEGCAQRERPGVYTNVVEYVDWILEKTQA |
| | | V |

All publications, patents, patent applications or other documents cited herein are hereby incorporated by reference in their entirety for all purposes to the same extent as if each individual publication, patent, patent application, or other document was individually indicated to be incorporated by reference for all purposes.

What is claimed is:

1. A modified human Factor XIa (FXIa) polypeptide comprising a catalytic domain corresponding to amino acids 370 to 606 of SEQ ID NO:1 wherein the polypeptide specifically binds a catalytic domain directed FXIa inhibitor, wherein the polypeptide contains a first mutation that reduces catalytic activity, and wherein the polypeptide exhibits reduced interactions with thrombin or a platelet receptor relative to a full-length FXIa polypeptide having the sequence of SEQ ID NO:1.

2. The modified human FXIa polypeptide of claim 1, wherein the first mutation is located within the catalytic domain.

3. The modified human FXIa polypeptide of claim 1 or 2, wherein the first mutation inactivates the catalytic domain.

4. The modified human FXIa polypeptide of any one of claims 1-3, wherein the first mutation is at a position corresponding to position 557 of SEQ ID NO:1.

5. The modified human FXIa polypeptide of any one of claims 1-4, wherein the first mutation is a serine to alanine substitution at a position corresponding to position 557 of SEQ ID NO:1.

6. The modified human FXIa polypeptide of any one of claims 1-5, wherein the modified human FXIa polypeptide further comprises an apple domain and wherein the second mutation is located in the apple domain.

7. The modified human FXIa polypeptide of claim 6, wherein the modified human FXIa polypeptide further comprises a plurality of apple domains and wherein each of the apple domains comprises a mutation.

8. The modified human FXIa polypeptide of any one of claims 1-5, wherein the modified human FXIa, polypeptide does not comprise an apple domain.

9. The modified human FXIa polypeptide of any one of claims 1-8, wherein the modified human FXIa polypeptide does not interact with thrombin or platelet receptors.

10. The modified human FXIa polypeptide of any one of claims 1-9, wherein the modified human FXIa polypeptide does not comprise a dimerization domain.

11. The modified human FXIa polypeptide of any one of claims 1-10, wherein the modified human FXIa polypeptide has a cysteine to serine substitution at a position corresponding to position 482 of SEQ ID NO:1.

12. A modified FXIa polypeptide comprising the amino acid sequence of SEQ ID NO:2, wherein the modified human FXIa polypeptide does not comprise a dimerization domain.

13. The modified human FXIa polypeptide of any one of claims 1-12, further comprising a signal peptide.

14. The modified human FXIa polypeptide of claim 13, wherein the signal peptide is fused to the N-terminus of the modified human FXIa polypeptide.

15. The modified human FXIa polypeptide of claim 13 or 14, wherein the signal peptide comprises the amino acid sequence of SEQ ID NO:4.

16. The modified human FXIa polypeptide of any one of claims 13-15, wherein the modified human FXIa polypeptide comprises the amino acid sequence of SEQ ID NO:6.

17. The modified human FXIa polypeptide of any one of claims 13-15, wherein the modified human FXIa polypeptide comprises the amino acid sequence of SEQ ID NO:7.

18. The modified human FXIa polypeptide of any one of claims 1-17, wherein the modified human FXIa polypeptide has reduced procoagulant activity compared to a full-length FXIa polypeptide having the sequence of SEQ ID NO:1.

19. The modified human FXIa polypeptide of claim 18, wherein the modified human FXIa polypeptide lacks anticoagulant activity.

20. The modified human FXIa polypeptide of any one of claims 1-19, wherein the FXIa inhibitor is a polypeptide, a peptidomimetic, an antibody, a small molecule, or a nucleic acid.

21. The modified human FXIa polypeptide of claim 20, wherein the FXIa inhibitor is an antibody.

22. The modified human FXIa polypeptide of claim 20 or 21, wherein the FXIa inhibitor selectively binds FXIa over FXI.

23. An isolated nucleic acid molecule comprising a nucleotide sequence encoding the modified human FXIa polypeptide of any one of claims 1-22.

24. The isolated nucleic acid molecule of claim 23, wherein the nucleic acid molecule comprises the nucleotide sequence of SEQ ID NO: 3.

25. An isolated nucleic acid molecule comprising a nucleotide sequence encoding the modified human FXIa polypeptide of any one of claims 13-17, wherein the nucleic acid molecule comprises the nucleotide sequence of SEQ ID NO:8.

26. A vector comprising the nucleic acid molecule of any one of claims 23-25.

27. An isolated host cell comprising the vector of claim 26.

28. An isolated host cell that produces the modified human FXIa polypeptide of any one of claims 1-22.

29. A method of producing a modified human FXIa polypeptide. comprising culturing the host cell of claim 27 or 28 under conditions that result in production of the modified human FXIa polypeptide, and isolating the modified human FXIa polypeptide from the host cell or culture.

30. A method for decreasing anticoagulant activity in a subject being administered an FXIa inhibitor, comprising administering to the subject a modified human FXIa polypeptide of any one of claims 1-22, thereby decreasing the anticoagulant activity in the subject.

31. A method for reducing clotting time in a subject being administered an FXIa inhibitor, comprising administering to the subject a modified human FXIa polypeptide of any one of claims 1-22, thereby reducing the clotting time in the subject.

32. The modified human FXIa polypeptide of any one of claims 1-22 for use in decreasing anticoagulant activity in a subject being administered an FXIa inhibitor.

33. The modified human FXIa polypeptide of any one of claims 1-22 for use in reducing clotting time in a subject being administered an FXIa inhibitor.

34. Use of the modified human FXIa polypeptide of any one of claims 1-22 in the manufacture of a medicament for decreasing anticoagulant activity in a subject being administered an FXIa inhibitor.

35. Use of the modified human FXIa polypeptide of any one of claims 1-22 in the manufacture of a medicament for reducing clotting time in a subject being administered an FXIa inhibitor.

36. A pharmaceutical composition comprising the modified human FXIa polypeptide of any one of claims 1-22, and a pharmaceutically acceptable excipient.
